(19) United States
(12) Patent Application Publication
(10) Pub. No.: US 2026/0315528 A1
(43) Pub. Date: Oct. 15, 2026

(54) MACROPHAGE THERAPY

(71) Applicant: THE UNIVERSITY COURT OF THE UNIVERSITY OF EDINBURGH, Edinburgh (GB)

(72) Inventor: Stuart FORBES, Edinburgh (GB)

(73) Assignee: THE UNIVERSITY COURT OF THE UNIVERSITY OF EDINBURGH, Edinburgh (GB)

(21) Appl. No.: 19/484,263

(22) PCT Filed: Mar. 26, 2024
(86) PCT No.: PCT/GB2024/050805

§ 371 Date: Nov. 13, 2025

(30) Foreign Application Priority Data
May 16, 2023 (GB) 2307295.2
Sep. 27, 2023 (GB) 2314788.7

(51) Int. Cl. A61K 35/15; A61P 1/16

(52) U.S. Cl. CPC A61K 35/15; A61P 1/16

(57) ABSTRACT

The present invention relates to methods of treating liver disease using compositions comprising macrophages.

### FIELD OF THE INVENTION

The present invention relates to methods of treating liver disease using compositions comprising macrophages.

### BACKGROUND TO THE INVENTION

Liver cirrhosis is a major health problem. In the UK, liver disease is the fifth leading cause of mortality, with over one million deaths in 2010 was estimated to be as a result of liver cirrhosis. The disease is associated with a high level of morbidity due to the progressive tissue damage, fibrotic scarring and loss of liver function, and the only curative option for end-stage disease is liver transplantation. However, donor organ availability cannot meet demand, and often patients with end-stage liver disease are not eligible for transplantation. Those who do receive transplantation require lifelong immunosuppression with the increased health risks involved. In 2015, it was reported that 611 patients were on the active liver transplant list in the UK. Patients could be waiting up to two years to receive a liver transplant. At one year post-registration 17% of patients were still waiting on the liver transplant list. Likewise, in the US, there are many patients on the liver transplant waiting list (LTWL) who may never identify a donor organ. Around 1700 patients die on the waiting list or are de-listed due to worsening health each year, while around 6500 have been on the list for more than 6 months. Treatment of end stage liver disease is a huge concern globally. The pathology of liver cirrhosis can be driven by numerous causative agents, including high alcohol consumption, obesity, metabolic disorders, viral infections or autoimmune disease, resulting in the progressive loss of healthy hepatocyte tissue and liver architecture, replaced by myofibroblast-derived fibrotic scarring. It has been increasingly recognised that if the agents driving liver damage are removed e.g. alcohol, viruses etc. then liver fibrosis can be at least partially reversible enabling liver regeneration to occur. Cirrhosis represents the end-stage of chronic liver injury and progressive fibrosis (scarring), irrespective of the underlying aetiology. It is characterised by severe liver fibrosis leading to architectural disruption, hepatocyte dysfunction and portal hypertension. Accordingly, many different underlying aetiologies may result in cirrhosis.

Despite the relative success of therapeutic interventions for specific aetiologies (e.g., novel antiviral therapy for hepatitis C virus infection, alcohol abstinence for alcoholic liver disease), many diseases (e.g. NASH) have not got approved medical therapies and patients often present to medical attention late when cirrhosis and related complications have already occurred [Starkey et al., Semin Liver Dis., 2019; 39 (4): 442-451]. Thus, there are no specific therapies to treat hepatic cirrhosis and so delivering an effective anti-fibrotic therapy is therefore a major unmet clinical need for both chronic and acute liver damage [Forbes & Newsome., Nat Rev Gastroenterol Hepatol. 2016; 13 (8): 473-85; Thomas et al., J Hepatol. 2018; 68, 1090-1091; Forbes et al., J Hepatol. 2015; 62, S157-169].

Hepatic decompensation (HD), defined by the acute development of one or more major complications of cirrhosis (i.e., ascites, encephalopathy, gastrointestinal variceal haemorrhage, and spontaneous bacterial peritonitis), represents a morbid advancement during the clinical course of liver cirrhosis [Trebicka et al., J Hepatol 2020; 73 (5): 1082-1091] and is the most common cause of hospitalisation in patients with liver cirrhosis [Moreau et al., 2013; 144 (7): 1426-37 1437.e1-9]. Patients with HD are at high risk for short-term death [Moreau et al., 2013; 144 (7): 1426-37 1437.e1-9]. The first episode of HD (also referred to herein as a first hepatic decompensation event), which often requires hospitalisation, signals the transition from compensated to decompensated cirrhosis. Decompensated cirrhosis is characterised by recurrent episodes of HD. HD has two distinct clinical presentations, depending on the presence or absence of other organ failures and the grade of systemic inflammation. The presence of multiple organ failures and high-grade systemic inflammation is the hallmark of acute-on-chronic liver failure (ACLF), a syndrome associated with a very high 28-day mortality rate. HD associated with moderate systemic inflammation not involving additional organs has a lower 28-day mortality rate (~2%, although this increases to 10% at 90-days suggesting a heterogeneity of clinical course in patients with HD) [Trebicka et al., J Hepatol 2020; 73 (5): 1082-1091] but still portends a poor outcome over the ensuing years.

Macrophages in the liver are a heterogeneous population of cells, including resident Kuppfer cells and recruited haematopoietic-derived macrophages, with diverse roles in the liver's regenerative response after injury such as phagocytosis, maintaining immune tolerance, both promotion and resolution of inflammation and fibrosis through activation of hepatic stellate cells/production of cytokines and degradation of the extracellular matrix.

During chronic liver injury models macrophages mediate the recruitment of proinflammatory cells and activation of hepatic stellate cells to promote fibrogenesis. Conversely, fibrosis regression is characterised by an in situ phenotypic switch to a restorative hepatic macrophage population with pro-resolution properties whereby liver repair and regeneration is facilitated by increased expression of matrix metalloproteinases (MMPs), growth factors and phagocytosis-related genes. This process of phenotypic ‘switching’ from a proinflammatory ‘M1-like’ to a pro-resolution ‘M2-like’ macrophage is mediated via down-regulation of NOD-containing, LRR-containing and pyrin domain-containing protein 3.

Macrophage-directed therapeutic approaches to liver diseases face a variety of challenges due to the complex nature of macrophage functions and interactions in the disease state. Previous trials have demonstrated concerns around cellular engraftment and expansive potential of such approaches. WO2019175595 and Moroni et al., (Nature medicine, 25 (10), 1560-1565.) both describe a single-arm phase I clinical trial of a macrophage therapy in the treatment of liver cirrhosis. This study was not designed or powered to demonstrate statistically significant changes in efficacy measures following macrophage therapy. Other clinical trials using macrophages as therapy have shown only transient benefits with no long-term improvement. (Zekri et al., Stem Cell Res Ther 2015; 6:1-14; Mohamadnejad et al., Liver Int 2013; 33:1490-6).

Alternative therapies are urgently required which prevent or delay the transition of liver disease to terminal decompensated stages, and which show long-term benefit.

### SUMMARY OF THE INVENTION

The invention provides a composition comprising macrophages, for use in the treatment of liver disease in a patient, wherein the treatment provides a greater reduction in symptoms than a treatment that does not comprise administration of macrophages. Liver disease is often a chronic disease with symptoms that can reduce or increase over time with or without treatment. Strikingly, the examples of the present application demonstrate that the macrophage therapies of the invention are effective to treat liver disease and provide a greater reduction in symptoms than a control treatment. This observation is revealed by the structure of the trial, using control patients.

The invention also provides a composition comprising macrophages, for use in preventing decompensation in a patient with compensated liver cirrhosis. Strikingly, the examples demonstrate that the macrophage therapies of the invention are effective for preventing decompensation. In particular, no patients receiving the macrophage therapy suffered decompensation, either over a year or after extended follow-up. In contrast, 2 decompensation events were observed in the control group.

The invention also provides a composition comprising macrophages, for use in preventing, or reducing the risk of, hepatic decompensation events in a patient having liver cirrhosis. The invention also provides a composition comprising macrophages, for use in preventing, or reducing the risk of, or reducing the incidence of, further decompensation events in a patient with liver cirrhosis who has undergone their first hepatic decompensation event, optionally in a patient with liver cirrhosis who has undergone and recovered from their first hepatic decompensation event, optionally wherein the first hepatic decompensation event required hospitalization. The patient who undergoes a hepatic decompensation event may exhibit one or more clinical signs of hepatic decompensation selected from the list consisting of jaundice, ascites, hepatic encephalopathy, hepatorenal syndrome, variceal haemorrhage and gastrointestinal haemorrhage. The compositions for use according to the invention are expected to be particularly effective in treating patients that have been hospitalised following their first hepatic decompensation event.

The invention also provides a composition comprising macrophages, for use in treating a patient with liver cirrhosis that has recovered from their first hepatic decompensation event (recompensated), optionally wherein the first hepatic decompensation event required the subject's hospitalization, preferably wherein the subject has not undergone an additional hepatic decompensation event after having recovered from the first decompensation event.

The invention also provides a composition comprising macrophages, for use in reducing the risk of death for a patient with liver disease. Strikingly, the examples demonstrate that the macrophage therapies of the invention are effective for reducing the risk of death. In particular, no patients receiving the macrophage therapy died during the year-long trial or after extended follow-up. In contrast, 3 patients in the control group died during the year-long trial and another during the follow up period. In certain embodiments, the composition is for use in increasing survival, such as transplant free survival, in a patient with liver disease.

The invention also provides a composition comprising macrophages, for use in reducing the risk of end stage renal disease in a patient with liver disease. Strikingly, the examples demonstrate that the macrophage therapies of the invention are effective for reducing the risk of end stage renal disease. In particular, no patients receiving the macrophage therapy developed end-stage renal disease during the year-long trial or after extended follow-up. In contrast, end stage renal disease was observed in the control group.

The invention also provides a composition comprising macrophages, for use in reducing adverse hepatobiliary events in a patient with liver disease, such as serious adverse hepatobiliary events. Strikingly, the examples demonstrate that the macrophage therapies of the invention are effective for reducing adverse hepatobiliary events. In particular, only 4 patients receiving the macrophage therapy experienced adverse events, and there were no serious events. In contrast, 9 adverse events, 3 of which were serious, were observed in the control group.

The invention also provides a composition comprising macrophages, for use in reducing the risk of disease progression. Strikingly, the examples demonstrate that the macrophage therapies of the invention are effective for stabilising liver disease and markedly reducing the number of patients experiencing disease progression, in particular more pronounced disease progression.

Therefore, the therapies of the invention are effective for reducing the risk of disease progression for each individual patient. Disease progression may be measured by MELD score or delta MELD, indicating a change in MELD score. Liver disease is often a chronic disease with symptoms that can reduce or increase over time with or without treatment. Although some patients may exhibit a reduction in MELD score (negative delta MELD), even without any treatment, the examples show that the macrophage treatments of the invention are effective for reducing the number of patients experiencing disease progression, and therefore, effective for reducing the risk of disease progression for each individual patient. Treatment may decrease the time taken for a patient to reach their maximum MELD score, as described in Example 5 herein, indicating that treatment may not only reduce disease progression but promote more rapid patient stabilisation. This example further suggests that treatment using the macrophage composition described herein may be useful for slowing liver disease progression, in particular liver cirrhosis, wherein slowing disease progression may be measured by a delay in increase in MELD score. Therefore, the invention provides a new treatment option, for clinical scenarios wherein any risk of disease progression is unacceptable.

In all aspects of the invention, the compositions comprising macrophages are for use in providing an extended therapeutic effect in the treatment of liver disease. In particular, the treatment may prevent decompensation, reduce the risk of death, reduce the risk of end stage renal disease, reduce adverse hepatobiliary events, and/or reduce disease progression, over a period of 3, 6, 9, 12, 14, 18 or 24 months following final administration of the composition. Strikingly, the examples demonstrate that the macrophage therapies of the invention are effective for providing therapeutic effects of extended duration. In particular, therapeutic effects including MELD score reductions, limitation of MELD score increases, reduction in decompensation, reduction in deaths, and reduction in adverse hepatobiliary events were observed after 90 days, 360 days and even after extended follow up at 430 days. These results are particularly surprising, considering that administered macrophages would be expected to be cleared from the body within 1-2 weeks of treatment. Furthermore, the therapeutic effect described in WO2019/175595 appears to peak at only 90 days, with delta MELD scores increasing from 90 days (see FIG. 9). The duration of therapeutic effect that is revealed in the examples provides new treatment option for patients with liver disease. Alternative therapies may be intended to provide some relief from symptoms or disease progression over a short period, until a suitable transplant can be arranged. The present invention allows patients with liver disease to be treated for longer periods.

In certain embodiments, the invention provides compositions comprising macrophages for use in use treating end-stage liver disease, optionally wherein the composition is for use in reducing the risk of death for a patient with end-stage liver disease over a period of 3, 6, 9, 12, 14, 18, 24, 30 or 36 months following final administration of the composition. The striking potency of the therapies tested in the examples provides a new treatment for end-stage liver disease, which otherwise has no treatment available. In addition, the duration of effect that is revealed in the examples provides a new treatment option for liver disease and end-stage liver disease in particular, because they can be treated without transplant for significant periods of time.

Accordingly, the invention also provides compositions comprising macrophages for use in treating liver disease in a patient scheduled to receive a liver transplant not sooner than 3, 6, 9, 12, 14, 18 or 24 months in the future following final administration of the composition. The striking duration of the therapeutic effect shown in the examples allows the treatment of patients that are not able to receive a liver transplant in the near future.

In any aspect of the invention, the treatment may comprise administration of at least one dose of the composition, such as at least two or at least three doses of the composition, or such as not more than one or not more than two or not more than three doses of the composition. In such embodiments, any therapeutic endpoints may be measured from the final administration of the composition, which may be a single administration.

The invention also provides compositions comprising macrophages for use in a method of treating liver disease in a patient, wherein the method comprises administering the composition and at least 3 months later, such as 6, 9, 12, 14, 18 or 24 months later, providing a liver transplant.

The invention also provides a method of treating a liver disease in a patient, wherein the method comprises administering a composition comprising macrophages.

The invention also provides a method of preventing decompensation in a patient with compensated liver cirrhosis, wherein the method comprises administering a composition comprising macrophages.

The invention also provides a method of preventing further decompensation events in a patient with liver cirrhosis who has undergone their first hepatic decompensation event, wherein the method comprises administering a composition comprising macrophages. According to some embodiments, preventing further decompensation events comprises preventing the patient from exhibiting one or more clinical signs of hepatic decompensation selected from the list consisting of jaundice, ascites, hepatic encephalopathy, hepatorenal syndrome, variceal haemorrhage and gastrointestinal hemorrhage, optionally wherein the one or more clinical signs requires hospital admission.

The invention also provides a method of treating a patient that has recovered from their first hepatic decompensation event (recompensated), optionally wherein the first hepatic decompensation event required the subject's hospitalization, preferably wherein the subject has not undergone an additional hepatic decompensation event after having recovered from the first decompensation event, wherein the method comprises administering a composition comprising macrophages.

The invention also provides a composition comprising macrophages, for use in treating a patient exhibiting one or more clinical signs of hepatic decompensation selected from the list consisting of jaundice, ascites, hepatic encephalopathy, hepatorenal syndrome, variceal haemorrhage and gastrointestinal haemorrhage, where the one or more clinical signs require hospital admission.

The invention also provides a method of reducing the risk of death for a patient with liver disease, wherein the method comprises administering a composition comprising macrophages.

The invention also provides a method of reducing the risk of end stage renal disease in a patient with liver disease, wherein the method comprises administering a composition comprising macrophages.

The invention also provides a method of reducing adverse hepatobiliary events in a patient with liver disease, wherein the method comprises administering a composition comprising macrophages.

The invention also provides a method of treating a liver disease in a patient, wherein the method comprises administering a composition comprising macrophages and wherein the method reduces the risk of disease progression over a period of 3, 6, 9, 12, 14, 18 or 24 months following final administration of the composition.

The invention also provides a method of treating end-stage liver disease, optionally wherein the end-stage liver disease is liver cirrhosis, optionally wherein the liver cirrhosis is compensated or decompensated liver cirrhosis, wherein the method comprises administering a composition comprising macrophages.

The invention also provides a method of treating end-stage liver disease, wherein the method comprises administering a composition comprising macrophages, and optionally wherein method reduces the risk of death for a patient with end-stage liver disease over a period of 3, 6, 9, 12, 14, 18, 24, 30 or 36 months following final administration of the composition. The invention also provides a method of treating liver disease in a patient, wherein the method comprises administering a composition comprising macrophages, and wherein the patient is scheduled to receive a liver transplant not sooner than 3, 6, 9, 12, 14, 18 or 24 months in the future following final administration of the composition.

The invention also provides a method of treating liver disease in a patient, wherein the method comprises administering a composition comprising macrophages, and wherein the method additionally comprises providing a liver transplant at least 3 months later, such as 6, 9, 12, 14, 18 or 24 months later. In related embodiments, the invention provides a method of treating liver disease in a patient, wherein the method comprises administering a composition comprising macrophages, and wherein the method additionally comprises providing advice that the patient is suitable to receive a liver transplant at least 3 months later, such as 6, 9, 12, 14, 18 or 24 months later.

The invention also provides a method of treating liver disease in a patient, wherein the treatment reduces the risk of death over a period of 3, 6, 9, 12, 14, 18, 24 or 36 months following final administration of the composition.

The invention also provides a method of treating liver disease in a patient, wherein the treatment increases the long-term survival of patients with inflammatory liver disease for a period of at least 1 year, optionally at least 2 years, preferably at least 3 years. In some embodiments, the inflammatory liver disease is liver cirrhosis.

The invention also provides a method of treating liver disease in a patient, wherein the treatment reduces all-cause mortality for a period of at least 1 year, optionally at least 2 years, preferably at least 3 years. In some embodiments, the liver disease is liver cirrhosis.

The invention also provides a method of treating liver disease in a patient, wherein the treatment reduces need for liver transplant for a period of at least 1 year, optionally at least 2 years, preferably at least 3 years. In some embodiments, the inflammatory liver disease is liver cirrhosis.

The macrophages used according to the invention may be unpolarized. The examples demonstrate striking therapeutic effects for therapies using unpolarized macrophages.

The macrophages used according to the invention may be polarized. Preferably, the macrophages are polarized and exhibit a pro-restorative M2-like phenotype. Therapies using M2-like cells are expected to be similarly, if not more, effective than unpolarized macrophages. Chronic liver injury models illustrate the role of proinflammatory cells in promoting fibrogenesis, and that repair is associated with a switch to an anti-inflammatory, M2-like phenotype. M2-like macrophages would be expected to be particularly efficacious in promoting liver repair due to the enhanced expression of matrix metalloproteinases (MMPs), growth factors and phagocytosis-related genes to resolve pathological inflammation. Accordingly, polarised, M2-like cells would be expected to show even greater efficacy in treating liver disease in a patient.

Preferably, the macrophages used according to the invention are autologous. For treatment of humans, the macrophages are human macrophages.

In some embodiments the human macrophages for use in accordance with any aspect or embodiment of the invention are monocyte-derived. In some embodiments the human macrophages for use in accordance with any aspect or embodiment of the invention are mature macrophages. Suitably, the macrophages for use in accordance with the invention are characterised by high or elevated expression of at least one macrophage-associated surface marker such as 25F9 or CD206; in some embodiments, expression is “high” or “elevated” in comparison to the expression level of these markers on the precursor or source cell such as freshly-isolated peripheral blood monocytes. Other surface markers include presence and/or high expression of one or more of CD163 or CD169. In some embodiments, absence of CD93 and a decrease in the inflammatory cytokine receptor CCR2, compared to isolated CD14+ monocytes on day 0, may be detected. In particular embodiments, unpolarised macrophages are characterised by positive expression of CD45/CD14, have viability greater than 80% and have a MFI (mean fluorescence intensity) for the surface markers 25F9 and CD206 more than 5 fold higher than the MFI of the original monocytes at day 0.

In one embodiment, autologous isolated human macrophages for use in accordance with any aspect or embodiment of the invention are prepared from CD14+ monocytes isolated from peripheral blood of a diseased patient. Suitably these CD14+ monocytes are incubated with M-CSF for at least 48 hours, or for 3 to 5 days, or 7 days. In some embodiments, M-CSF is used at a concentration of approx. 100 ng/ml although it will be appreciated that this amount may be varied to obtain unpolarized macrophages having the desired characteristics.

In some embodiments, the autologous isolated human macrophages in accordance with the invention are for use in the treatment liver cirrhosis, preferably wherein liver cirrhosis is caused by any of high alcohol consumption, obesity, metabolic disorders or viral infections, most preferably alcohol-induced liver cirrhosis, NASH or HCV. In some embodiments, the liver cirrhosis is compensated liver cirrhosis. In some embodiments, the liver cirrhosis is decompensated liver cirrhosis.

In other embodiments, autologous isolated human macrophages are for administration intravenously. Suitably the dose is from approximately 10⁷ to 10⁹ cells, suitably at least 10⁷ cells, at least 10⁸ or at least 10⁹ cells. Multiple doses may be administered.

In another aspect of the invention, the composition comprising macrophages is for use in a method of treating fibrosis, preferably cirrhosis, by administration of one or more doses of said macrophages to the patient in need thereof. In one embodiment, 1, 2, 3 or more doses of said macrophages are administered to a human in need thereof wherein there is an interval of approximately one month between each of said doses. In one embodiment, the first dose is on day 1, the second dose is on day 30 and the third dose is on day 60. In some embodiments, one or more doses are administered to a subject wherein there is an interval of approximately one year between each dose. In one embodiment, the macrophages are comprised in a pharmaceutical composition. In one embodiment administration of said macrophages results in a reduction in fibrosis. In one embodiment, administration of said macrophages results in a decrease in MELD score. In one embodiment, administration of said macrophages reduces the time taken for a patient to reach their maximum MELD score within a certain time period (e.g. a year) as compared to patients who have not been administered with the composition. In one embodiment, treatment results in the patient reaching their maximum MELD score in less than 50 days. In one embodiment, treatment results in the MELD score ceasing to increase or decreasing in less than 50 days.

In some embodiments, the patient has cirrhotic liver disease. Suitably, the human is a diseased patient having a MELD score of 10 to 16.

The number of macrophages in each dose may be varied according to the individual to be treated. In some embodiments, each dose comprises at least 1×10⁷ macrophages, suitably 1×10⁸ or 1×10⁹ macrophages per dose.

In one embodiment, administration of each dose is by intravenous administration. Suitably, intravenous administration is via a peripheral vein.

In some embodiments, the macrophages for use in a method in accordance with this aspect of the invention are in an unpolarized state. Suitably such macrophages are characterised by high expression of 25F9 or CD206 over that seen on the monocytes from which they are derived. In some embodiments, the unpolarized macrophages are derived from the human's own PBMCs i.e. are autologous. In other embodiments, the unpolarized macrophages may be allogeneic, e.g. derived from a healthy donor for administration to a patient with a relevant disease. In other embodiments, the unpolarized macrophages may be derived from stem cells bone marrow (BM), embryonic stem cells (ESC) or induced pluripotent stem cells (iPSC). Methods for generating suitable macrophages are described herein.

In another aspect, the invention provides a composition comprising autologous macrophages for use in a method of treating cirrhosis by intravenous administration of 3 doses of up to 10⁹ cells to a human having a MELD score of 10 to 16. Suitably these doses are provided approximately one month apart or at approximately 30 day intervals. In some embodiments, a use comprising autologous macrophages in accordance with the invention may be combined with another agent used in the treatment of liver cirrhosis or with an anti-fibrotic agent. In some embodiments, the other agent may be G-CSF, such as recombinant human G-CSF.

In other embodiments, the macrophages for use in a method according to the invention are in a polarised state. Preferably, said polarised macrophages have a pro-restorative M2-like phenotype and are anti-inflammatory and anti-fibrotic.

In preferred embodiments, the invention provides a composition comprising unpolarized autologous human macrophages, for use in:
treatment of liver disease in a patient, wherein the treatment provides a greater reduction in symptoms than a treatment that does not comprise administration of macrophages;
preventing decompensation in a patient with compensated liver cirrhosis;
preventing further decompensation events in a patient with decompensated liver cirrhosis who has undergone their first hepatic decompensation event;
treating a patient that has recovered from their first hepatic decompensation event (recompensated), optionally wherein the first hepatic decompensation event required the subject's hospitalization, preferably wherein the subject has not undergone an additional hepatic decompensation event after having recovered from the first decompensation event;
treating a patient exhibiting one or more clinical signs of hepatic decompensation selected from the list consisting of jaundice, ascites, hepatic encephalopathy, hepatorenal syndrome, variceal haemorrhage and gastrointestinal haemorrhage, optionally where the one or more clinical signs require hospital admission;
reducing the risk of death for a patient with liver disease;
reducing the risk of end stage renal disease in a patient with liver disease;
reducing adverse hepatobiliary events in a patient with liver disease, such as serious adverse hepatobiliary events;
reducing the risk of disease progression;
reduce the risk of death, reduce the risk of end stage renal disease, reduce adverse hepatobiliary events, and/or reduce disease progression, over a period of 3, 6, 9, 12, 14, 18 or 24 months following final administration of the composition;
treating end-stage liver disease, optionally wherein the composition is for use in reducing the risk of death for a patient with end-stage liver disease over a period of 3, 6, 9, 12, 14, 18, 24, 30 or 36 months following final administration of the composition
treating liver disease in a patient scheduled to receive a liver transplant not sooner than 3, 6, 9, 12, 14, 18 or 24 months in the future following final administration of the composition; and/or
treating liver disease in a patient, wherein the method comprises administering the composition and at least 3 months later, such as 6, 9, 12, 14, 18 or 24 months later, providing a liver transplant.

In preferred embodiments, the invention provides a composition comprising polarized autologous human macrophages, such as macrophages that exhibit a pro-restorative M2-like phenotype, for use in:
treatment of liver disease in a patient, wherein the treatment provides a greater reduction in symptoms than a treatment that does not comprise administration of macrophages;
preventing decompensation in a patient with compensated liver cirrhosis;
preventing further decompensation events in a patient with decompensated liver cirrhosis who has undergone their first hepatic decompensation event;
treating a patient that has recovered from their first hepatic decompensation event (recompensated), optionally wherein the first hepatic decompensation event required the subject's hospitalization, preferably wherein the subject has not undergone an additional hepatic decompensation event after having recovered from the first decompensation event;
treating a patient exhibiting one or more clinical signs of hepatic decompensation selected from the list consisting of jaundice, ascites, hepatic encephalopathy, hepatorenal syndrome, variceal haemorrhage and gastrointestinal haemorrhage, optionally where the one or more clinical signs require hospital admission;
reducing the risk of death for a patient with liver disease;
reducing the risk of end stage renal disease in a patient with liver disease;
reducing adverse hepatobiliary events in a patient with liver disease, such as serious adverse hepatobiliary events;
reducing the risk of disease progression;
reduce the risk of death, reduce the risk of end stage renal disease, reduce adverse hepatobiliary events, and/or reduce disease progression, over a period of 3, 6, 9, 12, 14, 18 or 24 months following final administration of the composition;
treating end-stage liver disease, optionally wherein the composition is for use in reducing the risk of death for a patient with end-stage liver disease over a period of 3, 6, 9, 12, 14, 18, 24, 30 or 36 months following final administration of the composition;
treating liver disease in a patient scheduled to receive a liver transplant not sooner than 3, 6, 9, 12, 14, 18 or 24 months in the future following final administration of the composition; and/or
treating liver disease in a patient, wherein the method comprises administering the composition and at least 3 months later, such as 6, 9, 12, 14, 18 or 24 months later, providing a liver transplant.

### BRIEF DESCRIPTION OF THE FIGURES

FIG. 1. The average delta-MELD (ΔMELD) score for all patients 90 days after treatment relative to baseline, in the control, 1 infusion and 3 infusion groups.

FIG. 2. The average maximum and minimum change in ΔMELD score in the control and 1 infusion groups 90 days after treatment.

FIG. 3. The individual ΔMELD scores for each patient in the 1 infusion (top panel) and control (bottom panel) groups 90 days after treatment.

FIG. 4. The average maximum and minimum change in ΔMELD score in the control and 1 infusion groups 360 days after treatment.

FIG. 5. The frequency of clinical events in the 1 infusion group and control group, during the 1 year trial, and during the extended follow-up period (430 days after treatment).

FIG. 6. The frequency of hepatobiliary disorder adverse events (AE) and serious adverse events (SAE) 360 days after treatment, in the treatment (n=23) and control (n=24) groups.

FIG. 7. Formulae for calculating MELD score (top), ΔMELD (middle) and delta-delta-MELD (ΔΔMELD, bottom).

FIG. 8. Overview of trial pathway following randomisation to the respective arms of the MATCH Phase II trial.

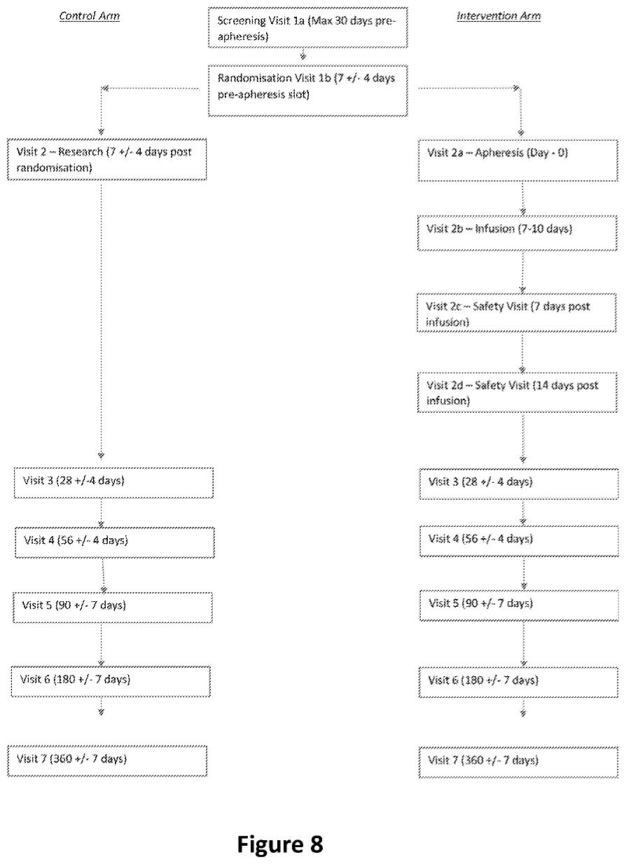

FIG. 9. The average length of time (days) for treated (1 infusion) and control groups to reach the maximum recorded MELD score.

FIG. 10. The length of time (days) for the individual patients in the treated (1 infusion) and control groups to reach the maximum recorded MELD score.

FIG. 11. (A) 1- and (B) 3-year Kaplan Meier survival on control versus cell treatment (1 or 3 doses merged)

FIG. 12. Comparison between of patient deaths and transplanted between control and cell treatment at 3-years follow up.

FIG. 13. MSD Analysis of pro-inflammatory (IL-1B, IL-6 and TNF-α) and MAS pathogenic cytokine (IL-8) concentrations following treatment with macrophage cell therapy.

FIG. 14. 1-year dMELD analysis of control versus cell treatment (one dose) of (A) full dataset or (B) removal of recidivist drinker on the cell treatment group. Analysis using a mixed-effect model with a Tukey multiple comparison test.

### DETAILED DESCRIPTION OF THE INVENTION

### Macrophages

Macrophages are phagocytic cells which have two distinct derivations-tissue resident macrophages which are derived from foetal cells, and found distributed throughout all tissues in the body, and peripheral blood monocyte-derived macrophages, which can differentiate in response to signals such as injury, inflammation and disease. Monocyte-derived macrophages have a spectrum of functions dependent upon context, and are involved in control of infectious organisms, clearance of damaged cells and repair of tissues. Early categorization of macrophages into M1 (classically-activated, inflammatory) and M2 (alternatively-activated, anti-inflammatory) is thought to be an over simplification, and macrophages can have both inflammatory and pro-resolution functions depending upon activation method and cytokine milieu, as described for example in Murray et al. (Immunity 2014; 41:14-20)) and Tacke et al. (J. Hepatology 2017; 66:1300-1312).

In the liver, macrophages can promote fibrogenesis both by activating the pro-fibrotic cytokine Transforming Growth Factor (TGF)-beta and by stimulating myofibroblast proliferation via platelet-derived growth factor (PDGF), IF-1beta and tumor necrosis factor-alpha. They are also critical for fibrosis resolution because they provide a rich source of the scar-degrading matrix metalloproteinases (MMPs). They produce factors such as MMP-9, which promote hepatic stellate cell apoptosis, needed for scar resolution. They also phagocytose cellular debris, which removes potential pro-inflammatory signals. The wide variety of responses is reviewed, for example by Tacke et al. (J. Hepatology 2017; 66:1300-1312).

Cirrhotic donors have high numbers of circulating monocytes, commonly >30% of the total nucleated cells (TNC) which is considerably more than reported values for healthy donors or the patients donating for CD14 processing (Campbell et al. Methods Mol Med. 2005; 109:55-70) and there is a close association with this and disease progression (Zimmermann et al. PFOS One 2010; 5 (6): e 11049). The data presented herein reports no significant changes between the functionality of macrophages derived from cirrhotic patients and healthy volunteers which indicates that the ex vivo maturation of circulating monocytes can be a technique to produce therapeutic mature macrophages for clinical cell therapy. Advantageously therefor a use of autologous cells i.e. the cirrhotic patient's own cells, is provided which minimises the risks of bio-incompatibility and immunological reactions from cell therapy. In addition, very high starting numbers of target cells can potentially reduce overall selection efficiency due to overwhelming the capacity of reagent and selection columns. Despite the high monocyte numbers in cirrhotic patients, the CliniMACS Prodigy system was surprisingly able to reproducibly isolate sufficient cells to meet a manufacturing criteria of a yield of at least 40% CD14+ cells.

As described in WO2019175595, the monocytes are cultured in GMP compliant, antibiotic-free defined media generating approximately 45% conversion rate to macrophages.

The macrophages used in accordance with the invention may be “polarized” or “unpolarized”. Macrophages may acquire various states, referred to as “polarisation”, which are usually, but simplistically, divided into two main extremes, “pro-inflammatory” (or classically-activated, “M1”, “M1-like”) and “pro-regenerative” (or “pre-restorative”, alternatively-activated, “M2”, “M2-like”, anti-inflammatory or anti-fibrotic). However, macrophages may adopt a state between these extremes which may be “unpolarised”, resting or naïve (M0) or point more towards an anti or pro-inflammatory state.

In some embodiments, the macrophages used in accordance with the invention may be engineered. In some embodiments, the macrophages for engineering have been produced from any suitable progenitor cell. In some embodiments, the macrophages have been produced in vitro.

In some embodiments, the macrophages for engineering are monocyte-derived. Preferably, the macrophages they are human monocyte derived macrophages (hMDM). Monocyte-derived means macrophages differentiated from monocytes. Monocytes are the natural precursors of macrophages and dendritic cells; they are contained in blood and bone marrow. In some embodiments, the macrophages are derived from peripheral blood monocytes, suitably the macrophages are peripheral blood monocyte derived macrophages. In some embodiments, the macrophages are human peripheral blood monocyte derived macrophages. In some embodiments, the monocytes are isolated from a human subject. In preferred embodiments, the monocytes are isolated from the human subject to be treated. In some embodiments, the macrophages are derived from the monocytes by culturing the monocytes, preferably in vitro. The macrophages may be derived from the monocytes by using any suitable culturing method.

In some embodiments, the invention relates to a cell therapy product for inflammatory organ damage based on monocyte-derived macrophages genetically modified with payloads that induce a M2-like phenotype (also referred to as a pro-restorative phenotype).

### Unpolarized Macrophages

Suitably, “unpolarized macrophages” in accordance with the invention are mature macrophages (i.e. have fully differentiated from the source macrophage progenitor or precursor cells, such as monocytes) but unpolarized (i.e. have not received further stimulation to induce a particular functional capacity). Unpolarized macrophages may also be described as “naive” or “non-activated”.

In one embodiment of any aspect of the invention, unpolarized macrophages are monocyte-derived cells. Thus, unpolarized macrophages may be derived from peripheral blood mononuclear cells (PBMCs) removed, for example, by leukapheresis or from a blood sample, and through isolating and culturing mononuclear cells. Suitably, monocytes cells are CD14+ cells which can be isolated through selecting for CD14 expression. Suitable methods are described in WO2019175595 and Moroni et al. (Nature medicine, 25 (10), 1560-1565.), and may include magnetic microbead selection.

In some embodiments, the PBMCs are removed from the patient to be treated so as to obtain patient-derived monocytes from which autologous unpolarized macrophage can be generated. Suitable methods for removing PBMCs from patients are described, for example, herein. Advantageously using autologous cells may avoid problems encountered from non-autologous approaches such as the introduction of exogenous bacteria or viruses, bio-incompatibility and immune rejection. In other embodiments, unpolarized macrophages may be allogeneic i.e. derived from monocytes obtained from a healthy donor. Suitably the healthy donor is matched, for example matched for HLA type. Briefly, methods for generating unpolarized macrophages include culturing CD14+ monocytes isolated from PBMCs in the presence of M-CSF to generate mature macrophages. Suitably, the isolated monocytes may be incubated for at least 48 hours or more, such as 3 or more days, preferably 7 days. In some embodiments, the patient-derived PBMCs may be maintained for a period e.g. overnight before processing or culturing to produce mature unpolarized macrophages. In some embodiments, those isolated cells may be provided in culture bags.

In some embodiments, unpolarized macrophages are generated from patient-derived monocytes in accordance with the culture methods and conditions as described in the Examples section herein. In some embodiments, the culture media is any media suitable for generating those unpolarized macrophages from source cells. The media may be serum-free. Suitably the media is a chemically defined media such as Good Manufacturing Practice (GMP)-compliant defined media to which suitable concentration of M-CSF is added. In some embodiments, the M-CSF may be recombinant M-CSF, preferably recombinant human M-CSF. Advantageously, the media is a medium optimised for the cultivation and expansion of T cells rather than a media specifically optimised for macrophages. In one embodiment, the media is TexMACs (Miltenyi) or AIM-V (Thermofisher).

In one embodiment, autologous isolated human macrophages for use in accordance with any aspect or embodiment of the invention are prepared from CD14+ monocytes isolated from peripheral blood of a diseased patient. Suitably these CD14+ monocytes are incubated with M-CSF for at least 48 hours, or for 3 to 5 days, or 7 days. In some embodiments, M-CSF is used at a concentration of approx. 100 ng/ml although it will be appreciated that this amount may be varied to obtain unpolarized macrophages having the desired characteristics.

Suitably the macrophages are derived from the monocytes by culturing the monocytes, suitably in vitro. Suitably the macrophages are derived from the monocytes by using any suitable culturing method.

Suitably the macrophages are produced in vitro from monocytes by a culturing method lasting between 3 to 8 days optionally 4 to 8 days. Suitably the macrophages are produced in vitro from monocytes by a culturing method lasting between 3 to 7 days, notably 4 to 7 days, or 5 to 7 days. In one embodiment, the macrophages are produced in vitro from monocytes by a culturing method that lasts 3-5 days, 4 or 5 days, or 7 days, known as a day 5 method or a day 7 method, respectively. One example of an in vitro method of producing macrophages from monocytes is described in WO2019/175595. The ‘day 5’ method is described in application number PCT/GB2021/051294 (the contents of which is herein incorporated by reference).

Suitably, the macrophages may be produced by a ‘day 5’ method comprising:
(a) Culturing monocytes in medium for 3-5 or 4-5 days to produce macrophages, wherein the medium comprises one or more growth factors to stimulate macrophage production; wherein step (a) takes place entirely in the same medium.

In some embodiments, the medium comprises one or more growth factors selected from the CSF family, preferably M-CSF. In some embodiments, the medium contains M-CSF at a concentration of between 25-150 ng/mL or 50-150 ng/mL. In some embodiments, the medium contains 100 ng/mL GMP-graded recombinant human macrophage colony-stimulating factor 1 (rhM-CSF-1; also known as ‘rh (recombinant human) CSF-1’). In some embodiments, the method for preparing autologous unpolarized macrophages generates a population of unpolarized macrophages having a viability of greater than 80% wherein viability may be measured by methods familiar to those skilled in the art such as DRAQ7 staining as described herein.

Unpolarised mature macrophages may be characterised by an elevated expression of at least one or both surface markers “25F9” and CD206. By “elevated” expression is meant expression which is increased in comparison to the expression of these markers in the source macrophage progenitor or precursor cells, such as monocytes (e.g. isolated day 0/untreated monocytes). In some embodiments, “elevated” expression means expression of surface markers that is at least approximately five-fold when compared with the source cells. Expression of markers can be determined by methods known to those skilled in the art including fluorescent labelling techniques as described herein. Suitably levels or expression may be measured by Mean Fluorescence Intensity (MFI) using methods as described herein. 25F9 refers to a macrophage marker antibody supplied by eBioscience/Thermofisher, which recognises a marker on mature macrophages but not immature macrophages or monocytes, or any other blood cell. The marker to which the antibody binds is unknown; the name derives from the antibody clone. Further details are given here https://www.theTOofisher.TOm/antibodv/product/Mafare-Macrophage-Marker-Antibody-clone-eBio25F9-25F9-Monoclonal/50-0115-41). In some embodiments, labels such as antibodies which compete with 25F9 for binding to the same surface marker may also be used to identify an unpolarized macrophage phenotype. As used herein, “expression of 25F9” means expression of the marker to which 25F9 binds.

In some embodiments, the source monocytes are CD45⁺, CD14⁻, 25F9, CD206. Incubation in a suitable media, such as a media containing M-CSF, generates unpolarized mature macrophages which are CD45⁺, CD14⁺, 25F9⁺, CD206⁺. In further embodiments, those source monocytes are also characterised by CCR2^{high}. CD163^{low}, CD169^{low}, maturing to unpolarized macrophages characterised by CCR2^{mid}, CD163⁺, CD169⁺.

Other markers whose expression may be increased in “unpolarized macrophages” include elevated levels of CD163 and CD169, and reduced levels of CCR2. In some embodiments unpolarized macrophages may be described as pro-resolution and/or anti-fibrotic by virtue of the expression of particular surface markers or scavenger receptors involved in clearance and uptake of damaged cells, fibrotic material and pro-inflammatory cytokines. Nomenclature of macrophages is reviewed, for example, in Martinez and Gordon (FIOOQPrime Rep. 2014; 6:13)

Unpolarized mature macrophages may also be derived from stem cells including embryonic stem cells (ES cells), human induced pluripotent stem cells (human iPSCs) and bone marrow-derived hematopoietic stem cells.

Various methods of producing macrophages from pluripotent stem cells such as ES cells and iPSCs are known in the art, see van Wilgenburg et al. (PLOS One 2013; 8 (8): e71098), for example. Methods for determining that a developed cell line is pluripotent and have a normal karyotype are described, for example in Yang et al. (Stem Cells 2017; 35 (4): 886-897). A suitable human induced pluripotent stem cell line includes SFCi55, described, for example in co-pending application PCT/GB2017/052769.

Suitable methods of generating macrophage progenitors from such a human iPS cell line may be as follows: They were maintained in StemPro medium prepared by supplementing DMEM/F12 with Glutamax (Invitrogen) with StemPro supplement (Invitrogen), 1.8% BSA (Invitrogen), 0.1 mM b-mercaptoethanol (Invitrogen) and 20 ng/ml human basic FGF (Invitrogen). The method for differentiation of iPSCs to macrophages was adapted from van Wilgenburg et al. On Day 0, spent medium was removed from one confluent well of a 6-well plate, and replaced with 2 ml StemPro (ThermoFisher) supplemented with cytokine Mix 1 (50 ng/ml BMP4, 50 ng/ml VEGF, and 20 ng/ml SCF). Cells were cut using the EZPassage™ tool, and gently dislodged with a Pasteur pipette. 5 They were divided equally into two wells of an Ultra-Low Attachment 6-well plate (Coming), and 2 ml X-VIVO™ 15 media with cytokine Mix 1 was added to each well. Cells were cultured in suspension for 3 days (with a cytokine top up on Day 2), to make embryoid bodies (EBs). On Day 4, EBs were lifted and transferred to gelatin-coated tissue-culture grade 6-well plates in X-VIVO™ 15 media supplemented with cytokine Mix 2 (100 ng/ml M-CSF, 25 ng/ml IL3, 2 mM Glutamax, 1% Penicillin/Streptomycin, 0.055 M b-mercaptoethanol). Approximately 30 EBs were plated in each well. EBs were maintained in this medium for the remaining duration of the protocol, with spent medium being replaced with fresh medium every 3-4 days. After about 2 weeks, the EBs produced macrophage progenitors in the culture supernatant and these were harvested and transferred to 10 cm² bacteriological dishes in X-VIVO™ 15 medium supplemented with cytokine Mix 3 (100 ng/ml M-CSF, 2 mM Glutamax, 1% Penicillin/Streptomycin) and allowed to mature for 5-7 days into iPSC-derived macrophages (iPSC-DM). Macrophage progenitors could be harvested twice a week for approximately 2 months.

Other methods for generating macrophage progenitors are described in Yeung et al. Sci. Rep. 2015; 5:8908, Zhuang et al. J Immunol Methods. 2012; 385 (1-2): 1-14, Sneju et al. Oncoimmunology 2014; 3 (1): e27927, Hale et al. PLOS One 2015; 10 (5): e0124307, Zhang et al. Circ Res. 2015; 117 (1): 17-28, Mucci et al. Stem Cell Reports 2016; 7 (2): 292-305 and van Wilgenburg et al. PLOS One 2013; 8 (8): e71098.

ES cell derived macrophages (ESDMs) may be generated by culturing the ES cells in the presence of colony stimulating factor-1 (CSF-1) (also known as M-CSF) and IL-3 to form embryoid bodies (EB). Whilst EBs adhere to tissue culture plastic, macrophage progenitor cells are non adherent and thus are released into the medium. The macrophage progenitor cells may then be harvested at various time points, for example after 10 or 20 days and plated onto non-treated Petri dishes and cultured in the presence of CSF-1 alone. This process can give rise to monocyte-like cells that adhere to the plastic forming a monolayer and mature into ESDM.

The maturation of the ES cells into ESDM can be monitored by detecting the presence of mature macrophage specific markers including 25F9 (human macrophage specific) and/or CDI lb. Additionally, human macrophages may be characterised by the absence of the monocyte marker CD93. Advantageously, the method described yields a substantially homogenous population of ESDM.

### Polarised Macrophages

In some embodiments, the macrophages for use according to the invention are polarised. Preferably, the polarised macrophages have an “M2” or pro-restorative phenotype, are anti-inflammatory and/or anti-fibrotic. In some embodiments, the macrophages are genetically engineered to induce an M2-like phenotype.

Polarised macrophages may be prepared using the same processes as discussed above for unpolarised macrophages, with additional steps to provide polarisation.

In some embodiments, unpolarized mature macrophages may be capable of being polarised into a M1 or M2-like phenotype by incubation with suitable polarising factors, for example M1 polarisation may be obtained by incubation with INF-gamma or TLR agonists such as LPS or poly (I:C), while M2 polarisation may be obtained by stimulating with IL4. In some embodiments, unpolarised macrophages are converted to an M2 phenotype upon exposure to macrophage colony-stimulating factor (M-CSF), IL-4, IL-13, IL-10 and TGF-β. These macrophages mediate wound healing and tissue regeneration primarily.

### Liver Diseases

In some aspects or embodiments the invention provides for treatment of a liver disease in a patient, wherein the treatment provides a greater reduction in symptoms than a treatment which does not comprise macrophages.

In some embodiments, a patient has suffered liver injury. In preferred embodiments, the liver injury is an inflammatory liver injury. In a preferred embodiment, the inflammatory liver injury has a fibrotic element. In preferred embodiments, the liver injury is a chronic liver injury, optionally a chronic, inflammatory liver injury. In preferred embodiments, the condition is a chronic inflammatory liver injury with a fibrotic element, preferably liver cirrhosis. In a preferred embodiment, the liver injury is cirrhosis. Chronic liver injury results in scar deposition and hepatocyte loss. Excessive accumulation of scar tissue results in liver fibrosis. At this stage, fibrosis can be reversed. However, untreated fibrosis can ultimately lead to cirrhosis.

As discussed herein, cirrhosis may result from diverse aetiologies. Hepatic disorders having a fibrotic component and therefore contribute to cirrhosis include, but are not limited to, non-alcoholic fatty liver disease (NAFL) (e.g., non-alcoholic fatty liver disease (NAFLD) or non-alcoholic steatohepatitis (NASH)) or alcoholic liver disease (e.g., alcoholic fatty liver disease (AFLD) or alcoholic steatohepatitis (ASH)). Fibrotic diseases, disorders, and conditions can include mechanical trauma, biliary obstruction, autoimmune hepatitis, iron overload, Hepatitis B infection (HBV), and/or Hepatitis C infection (HCV).

The aetiologies leading to fibrosis may include, but are not limited to, steatotic liver disease (SLD), such as metabolic dysfunction-associated steatotic liver disease (MASLD), Metabolic-associated steatohepatitis (MASH) or Met-ALD. In some instances, the cause of fatty liver disease may be unknown, and may be termed cryptogenic SLD. Metabolic dysfunction-associated steatotic liver disease refers to a non-alcoholic fatty liver disease, and therefore may also be known as NAFLD. Metabolic-associated steatohepatitis refers to a more severe form of MASLD, which may also be known as NASH. “Met-ALD” refers to individuals who have steatotic liver disease and who also drink alcohol. “Cryptogenic SLD” refers to SLD whose cause is unknown, such as in individuals who do not carry any known metabolic risk factors for SLD.

Cirrhosis may be either compensated or decompensated cirrhosis. Decompensated cirrhosis is defined as an acute deterioration in liver function in a patient with cirrhosis and is characterised by symptoms such as, but not limited to, jaundice, ascites, hepatic encephalopathy, hepatorenal syndrome, variceal haemorrhage, gastrointestinal haemorrhage and any combination thereof. In other embodiments, the liver disease is decompensated cirrhosis. In some embodiments, the liver disease is compensated cirrhosis. In some embodiments, the patient has compensated cirrhosis and had at least one event (or exactly one event) of decompensated cirrhosis. In a preferred embodiment, the patient has a MELD score of between 10-16 or 12-18 or 10-18.

In some embodiments, the liver disease is one in which cirrhosis is caused by damage to hepatocytes, for example, is a hepatocyte-derived disease, such as those diseases of viral origin (including treated (sustained viral response) hepatitis C (HCV), hepatitis B), damage through alcoholism (alcohol related liver disease (ALD)), or non-alcoholic fatty liver disease (NAFLD), including Non-alcoholic steatohepatitis (NASH) (including NASH resulting from diabetes or obesity), cryptogenic cirrhosis, haemochromotosis or alpha-1-antitrypsin deficiency. In some embodiments, the underlying aetiology has been removed (for example, a patient suffering from damage through alcoholism is no longer drinking, or a patient suffering from damage through HCV no longer has HCV etc.). In some embodiments, the patient with liver disease is at risk of end stage renal disease.

In some embodiments, the liver disease is steatotic liver disease (SLD). In some embodiments, the steatotic liver disease is metabolic dysfunction-associated steatotic liver disease (MASLD), is Met-ALD or cryptogenic SLD. In some embodiments, the metabolic dysfunction-associated steatotic liver disease is Metabolic-associated steatohepatitis (MASH).

Cirrhosis may lead to acute-on-chronic liver failure (ACLF). In some embodiments, the liver disease is ACLF. ACLF is a distinct condition from hepatic decompensation. Hepatic decompensation is characterised by the development of ascites, hepatic encephalopathy, gastrointestinal haemorrhage, or any combination of these conditions in patients with liver cirrhosis. ACLF in contrast is associated with organ failures and carries high short-term mortality in excess of 15% at 28 days. Three major features characterise this syndrome: ACLF occurs in the context of intense systemic inflammation; ACLF frequently develops in close temporal relationship with pro-inflammatory precipitating events (eg infections or alcoholic hepatitis); and ACLF is associated with single- or multiple-organ failure.

A diseased patient suitable for a treatment or use in accordance with any aspect or embodiment of the invention may be a patient with a relevant disease and severity.

In some embodiments, the patient has undergone their first hepatic decompensation event. In preferred embodiments, the patient has been hospitalised following their first hepatic decompensation event. The patient may exhibit one or more clinical signs of hepatic decompensation selected from the list consisting of jaundice, ascites, hepatic encephalopathy, hepatorenal syndrome, variceal haemorrhage and gastrointestinal haemorrhage. The compositions for use according to the invention are expected to be particularly effective in treating patients that have been hospitalised following their first hepatic decompensation event. Furthermore, the data provided in the examples demonstrate that the compositions for use according to the invention are suitable for treating these particular patients, which have severe disease that is difficult to treat.

Hospitalisation following a hepatic decompensation event is a measure of disease severity and provides a specific clinical situation. Certain symptoms of hepatic decompensation are similar to those of less severe liver cirrhosis, but when a patient is hospitalised following their first hepatic decompensation event, this indicates that their disease is severe enough to particularly benefit from the present invention.

Accordingly, in certain embodiments, the invention provides compositions for use in a treating a patient exhibiting or having recovered from one or more clinical signs of hepatic decompensation selected from the list consisting of jaundice, ascites, hepatic encephalopathy, hepatorenal syndrome, variceal haemorrhage and gastrointestinal haemorrhage, where the one or more clinical signs require hospital admission.

Hospitalisation refers to admission to a hospital for treatment. Hospitalisation thus normally requires that the patient stays in the hospital for at least 24 hours. Hospitalisation is a measure of disease severity, because symptoms cannot be managed outside of the hospital setting. Hospitalisation is a recognised measure of disease severity and patient status in the context of liver cirrhosis (see, for example, Balcar et al., United European Gastroenterol J. 2021; 9 (4): 427-437.)

In preferred embodiments, the patient is treated with composition once the patient has recovered from their first hepatic decompensation event (re-compensated), optionally wherein the patient was hospitalised following their first hepatic decompensation event. In some embodiments, the patient optionally is treated with the compositions for use according to the invention following discharge of the patient from the hospital. According to some embodiments, recovery from a hepatic decompensation event (re-compensation) is defined by a physician's clinical assessment and/or by no substantial elevation in MELD score between discharge from the hospital and treatment. In some embodiments, the patient exhibits and/or has recovered from one or more clinical signs of hepatic decompensation selected from the list consisting of jaundice, ascites, hepatic encephalopathy, hepatorenal syndrome, variceal haemorrhage, and gastrointestinal haemorrhage.

According to preferable embodiments, the patient is treated following recovery from a first hepatic decompensation event that required hospitalisation and prior to the undergoing additional hepatic decompensation events. As known in the art, the severity of decompensated cirrhosis and the mortality rate increases once a patient has had more than a single decompensation event [D'Amico et al., Journal of Hepatology, 2023 vol. 78 (S1) S105. Thus, without being bound by theory or mechanism, it is preferable to treat patients with the cells, compositions and cell populations of the invention following recovery from the first decompensated event to increase the chances of the patients not undergoing additional hepatic decompensation events and surviving without need for liver transplantation.

In a preferred embodiment, the liver disease is cirrhosis and the patient is treated following recovery from a first hepatic decompensation event that required hospitalisation, wherein the patient has recovered from one or more clinical signs of hepatic decompensation selected from the list consisting of jaundice, ascites, hepatic encephalopathy, hepatorenal syndrome, variceal haemorrhage and gastrointestinal haemorrhage, wherein the one or more clinical signs require hospital admission, and prior to the undergoing additional hepatic decompensation events.

### Treatment

The term “treatment” as used herein refers to preventing the progression of, and reducing partially or completely the clinical symptoms of, a patient with a liver disease or injury. Treatment with the composition comprising macrophages according to the invention provides a greater reduction in symptoms than a treatment that does not comprise administration of macrophages. In some embodiments, the treatment may result in an increase in or an acceleration of liver regeneration. Advantageously, the macrophages used in accordance with the invention may be anti-fibrotic and pro-regenerative cells.

Suitably, administration of unpolarized or polarised human macrophages in accordance with the invention may result in one of more of the following advantageous effects: a reduction in fibrosis or a reduction in liver disease in a subject, a reduction in necrosis, an increase in liver cell proliferation, a reduction in levels of pro-inflammatory cytokines, an increase in phagocytosis at the site of fibrosis.

In some embodiments, treatment reduces the risk of patients having major clinical complications associated with liver cirrhosis. In some embodiments, the major clinical complications of liver cirrhosis may be ascites, hepatic encephalopathy, gastrointestinal variceal haemorrhage, spontaneous bacterial peritonitis, jaundice, hepatorenal syndrome, gastrointestinal haemorrhage, death and a combination thereof.

In some embodiments, treatment prevents decompensation in a patient with compensated liver cirrhosis. In some embodiments, treatment prevents further decompensation events in a patient with liver cirrhosis who has undergone their first hepatic decompensation event, preferably wherein the first hepatic decompensation event required the patient's hospitalization, preferably wherein the patient has not undergone an additional hepatic decompensation event after having recovered from the first decompensation event. In some embodiments, the major clinical complication is death. In some embodiments, treatment reduces the risk of death in a patient with liver disease. In particular embodiments, treatment reduces the risk of death over a period of 3, 6, 9, 12, 14, 18 or 24 months following final administration of the composition. In some embodiments, the reduction in the risk of death is achieved after the administration of at least one dose of the composition. In one embodiment, the reduction in the risk of death occurs after the administration of one dose.

In some embodiments, the major clinical complication is end stage renal disease. In some embodiments, treatment reduces the risk of end stage renal disease in the patient with liver disease. In some embodiments, the reduction in the risk of end stage renal disease is achieved after the administration of at least one dose of the composition. In one embodiment, the reduction in the risk of end stage renal disease occurs after the administration of one dose.

In some embodiments, treatment reduces the risk of disease progression. In some embodiments, treatment prevents disease progression over a period of 3, 6, 9, 12, 14, 18 or 24 months following final administration of the composition. In some embodiments, disease progression is measured by a change in MELD score. In some embodiments, treatment limits the total change in MELD at 90 days to a total change of less than 2 units In some embodiments, treatment reduces the risk of the MELD score for the patient increasing by 1 or more, 1.5 or more, or 2 or more, optionally over a period of 3, 6, 9, 12, 14, 18 or 24 months following final administration of the composition. In some embodiments, treatment prevents disease progression over a period of 3, 6, 9, 12, 14, 18 or 24 months following final administration of the composition. In addition to limiting the overall change in MELD score, treatment may further reduce the time taken to achieve the maximum recorded MELD score within a certain time period (e.g. a year) as compared to patients who have not been administered with the composition. In some embodiments, the patient reaches the maximum recorded MELD score less than 50 days after treatment with a single infusion of the composition of the invention. Suitably, treatment may stabilise the patient with liver disease.

Accordingly, in some embodiments, treatment results in the MELD score ceasing to increase or decreasing in less than 50 days. Accordingly, in one aspect, the invention further provides a composition for use in a method of stabilising a patient with liver disease, wherein the method comprises administering a composition comprising macrophages. Stabilisation of a patient with liver disease may be determined by measuring MELD score. A patient may be deemed stabilised when their MELD score stops increasing significantly, i.e. when the maximum recorded MELD score is achieved. For example, a patient's MELD score may be measured monthly. When a patient is stabilised, the MELD score may not change significantly, in particular may not increase significantly, for a period of at least 3 months (i.e. 3 consecutive measurements give the same MELD score that does not change significantly), preferably at least 6 months, more preferably at least 12 months. In some embodiments, treatment reduces the length of time taken for the patient to be stabilised. Suitably, treatment reduces the length of time taken for maximum MELD score to be achieved, or for the MELD score to cease increasing or start decreasing. In some embodiments, treatment additionally reduces the extent of the increase of the MELD score. A significant change in MELD score is a change i.e. an increase or decrease, of at least 1 point of MELD, or at least 2 points of MELD. In some embodiments, stabilisation of the patient with liver disease may be determined by UKELD score.

Typically, liver disease is measured by the MELD (Measure of End-stage Liver Disease) score, a scoring system derived from serum markers blood bilirubin, creatinine and clotting potential. The scale was originally developed to predict mortality and is used to prioritize patients for liver transplantation. It predicts three month and one year mortality and predicts clinical decompensation in patients with compensated cirrhosis.

A change in MELD is a more significant determinant of death than initial MELD alone. For any given MELD, the magnitude and direction of change in MELD score during the previous 30 days is a significant independent mortality predictor. MELD score has been used by all the major Western regulatory authorities involved in liver transplantation (UK Transplant, Eurotransplant and UNOS) to help prioritise the allocation of liver transplants. In some embodiments, treatment with unpolarized or polarized human macrophages in accordance with the invention may result in a change in the MELD in a subject. In other embodiments, treatment may result in a change in one of the liver-related components of the MELD score i.e. bilirubin (BIL) and/or international normalized ratio (INR). In some embodiments, treatment reduces the risk of the MELD score for the patient increasing by 1 or more, 1.5 or more, or 2 or more, optionally over a period of 3, 6, 9, 12, 14, 18 or 24 months following final administration of the composition.

In other embodiments, treatment with unpolarized or polarized human macrophages in accordance with the invention may result in a change in UKELD. Other methods for measuring an effect in a subject include assessing changes in liver fibrosis as measured by transient elastography, by using an Enhanced Liver Fibrosis Test)-ELISA, by measuring liver metabolism and regenerative activity using labelled metabolites and a liver MRI scan or measuring liver volume and blood flow using MRI. In other embodiments, treatment with unpolarized or polarized human macrophages in accordance with the invention may result in a change of ELF or one of the liver-related components of ELF score i.e. hyaluronic acid (HA), tissue inhibitor of metalloproteinase-1 (TIMP-1) and propeptide of type III procollagen (PIIINP), In other embodiments, disease specific biomarkers and epitopes such as neoepitopes may be measured. For example, treatment with unpolarized or polarized human macrophages in accordance with the invention may result in a change in the precisely cleaved N-terminal propeptide of type III collagen (PRO-C3) and/or a peptide of helical collagen type III degradation (C3M). During fibrillar assembly the N-terminal propeptide of type III collagen is cleaved off by N-proteases but sometimes the removal of the propeptide is incomplete, thus PIIINP can be a marker of both fibrillogenesis formation and degradation, whereas PRO-C3 is a marker of formation as PRO-C3 is specific to the N-protease cleavage site. For example, ELF, liver related components of ELF, PRO-C3 and C3M may be measured in accordance with any of the following documents: Thiele et al. (Gastroenterology 2018; 154:1369-1379), Irvine K et al. (Liver International 2016, 370-377 ISSN 1478-3223), Barascuk N et al. (Clinical Biochemistry 34 (2010) 899-904), and Nielsen M J et al. (Am J Transl Res 2013; 5 (3): 303-315).

The term “subject” as used herein, refers to any individual who may benefit from the treatment of a liver injury. The subject may be a human subject, a human in need of a treatment, such as a diseased patient. Suitably a human suitable for a treatment in accordance with the invention is one with cirrhotic liver disease, preferably a patient having a MELD score of between 10 and 16. A human with a developing liver disease may also be suitable for such treatment, such as a human/patient with portal hypertension. The terms “patient” and “subject” may be used interchangeably herein.

In preferred embodiments, the patient has recovered from their first hepatic decompensation event (re-compensated), optionally wherein the patient was hospitalised following their first hepatic decompensation event. In some embodiments, the patient optionally is treated with the compositions for use according to the invention following discharge of the patient from the hospital.

### Dose

It will be appreciated that a therapeutically effective amount or dose of unpolarized or polarized macrophages will be dependent on various factors including the weight of the subject to be treated. By way of example, a therapeutically effective amount may be in the form of a dose of 1×10⁷ to 1×10⁹ unpolarized or polarized macrophages. In some embodiments, the dose will be multiples of 10⁷, 10⁸ or 10⁹ macrophages per dose. Suitably, the treatment will consist of three doses administered as infusions on a monthly basis i.e. 3 infusions of approximately up to 10⁹ macrophages at an interval of approximately 30 days. Advantageously, additional doses may enable further degradation of scar tissue to occur. In one embodiment, the macrophages for all doses to be administered to a patient would be collected through a single leukapheresis collection. In some embodiments, the final dose administered may depend upon the starting number of monocytes and can be multiples of 10⁸depending upon patient. In some embodiments, the treatment comprises the administration of one or more doses. In one embodiment, treatment comprises or consists of one dose.

In any aspect of the invention, the treatment may comprise administration of at least one dose of the composition, such as at least two or at least three doses of the composition, or such as not more than one or not more than two or not more than three doses of the composition. In such embodiments, any therapeutic endpoints may be measured from the final administration of the composition, which may be a single administration.

In some embodiments, the macrophages in accordance with the invention may be provided in a suitable storage or transfer bag.

### Administration

The macrophage product in accordance with any aspect of the invention can be administered into the body of the recipient by any suitable means, including but not limited to transdermally, subcutaneously, intramuscularly, parentally, enterally, intravenously, intraperitoneally, intraorbitally, intraretinally, by transplantation of tissue and into cerebrospinal fluid. Advantageously, the macrophage produce is administered intravenously such as, for example, intravenous injection or infusion.

Earlier work supports that portal vein delivery maximises the number of cells delivered to the liver, this route of delivery however, is not desirable in a clinical setting as repeated portal vein administration in cirrhosis patients risks portal hypertension and coagulopathy. The present inventors have found that although lower numbers of cells are likely to distribute to the liver following intravenous injection, this route of delivery has efficacy in the clinic.

In some embodiments, the unpolarized or polarized macrophages for use in accordance with the invention are prepared in a pharmaceutically acceptable carrier. A pharmaceutically acceptable carrier may be any substance which enables delivery of unpolarized or polarized macrophages to a subject and may include any suitable diluent or excipient or combination thereof. In some embodiments, unpolarized or polarized macrophages may be delivered in a saline solution supplemented with a human albumin solution.

Thus, the unpolarized or polarized macrophages for use in accordance with the invention may be provided in a pharmaceutical composition comprising a therapeutically effective amount of unpolarized or polarized macrophages and a pharmaceutically acceptable carrier.

In a suitable embodiment, a pharmaceutical composition of the invention may further comprise a pharmaceutically acceptable concentration of salt, buffering agents, and compatible carriers. The compositions may also include antioxidants and/or preservatives. In some embodiments a pharmaceutical composition may comprise DMSO, for example, 5-10% DMSO.

In preferred embodiments, the compositions for use according to the invention are administered to a patient that has recovered from their first hepatic decompensation event (recompensated), optionally wherein the first hepatic decompensation event required the patient's hospitalization, preferably wherein the patient has not undergone an additional hepatic decompensation event after having recovered from the first decompensation event.

### Combinations

In some embodiments, a macrophage treatment in accordance with the invention may be combined with another treatment for liver disease. Suitable treatments include treatment with G-CSF.

In some embodiments, the other treatment may comprise an anti-fibrotic drug treatment. Suitable antifibrotic drugs are reviewed, for example, by Wang et al. (Front Physiol. 2016; 7:47; doi: 10.3389/fphys.2016.00047) and Tacke et al. (J. Hepatology 2017; 66:1300-1312)).

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### EXAMPLES

Certain aspects and embodiments of the invention will now be illustrated by way of example and with reference to the figures described above.

### Example 1. Trial Design

### Study Oversight

The MATCH 0.1 trial was an investigator-led study, funded by the Medical Research Council (reference MR/M007588/1) and sponsored by ACCORD (Academic and Clinical Central Office for Research and Development for National Health Service (NHS) Lothian/University of Edinburgh). Trial oversight is also provided by a TSC and DMC, who are impartial around aspects of study design and logistics but provide independent advice and interval safety analyses. All study-related documents were designed by the trial team with input from ACCORD, an independent statistician and the Scottish National Blood Transfusion Service (SNBTS) team. The trial was conducted according to the ethical principles of the Declaration of Helsinki 2013 and was approved by Scotland A Research Ethics Committee (reference 15/SS/0121), NHS Lothian Research and Development department and the Medicine and Health Care Regula-tory Agency (MHRA-UK). Good Clinical Practice regulations were followed and written informed consent was obtained from all participants.

### Eligibility Criteria (Inclusions/Exclusions)

### Inclusion Criteria

Aged between 18 and 75 years (inclusive) at time of screening
Aetiology: One or more of:
   Alcohol-related liver disease (no active alcohol misuse 06 calendar months prior to screening).
   Features of chronic liver disease with a compatible history of alcohol excess (>80 g/day), in the absence of other causes of chronic liver disease.
   PBC 2 out of: Cholestatic liver function tests (LFTs), Positive antimitochondrial antibody (titre >1:40). Compatible liver histology (if already receiving ursodeoxycholic acid must be established on current dose >3 months prior to enrolment).
   NAFLD Either: Histological evidence of hepatic steatosis in the absence of other liver diseases Or: Imaging compatible with NAFLD (eg, fatty infiltration of liver) and one or more risk factors (eg, elevated body mass index, type 2 diabetes mellitus, hypertriglyceridaemia, hypertension) and the absence of significant alcohol consumption (<20 g/day) and no evidence of other causes of chronic liver disease.
   Cryptogenic cirrhosis diagnosis of cirrhosis unattributable to any other cause.
   Haemochromatosis diagnosis made on basis of compatible biochemistry (transferrin saturation >60%, ferritin >400), genotype (homozygous C282Y or H63D compound heterozygote) or histology.
   Alpha-1 antitrypsin deficiency diagnosis based on compatible genetic, phenotypic or histological testing.
   Previous chronic hepatitis C (sustained viral response i.e., undetectable HCV RNA 24 weeks after treatment).
Diagnosis of cirrhosis—invasive or non-invasive criteria cirrhosis defined as any of:
   Biopsy-confirmed diagnosis of cirrhosis.
   Transient elastography (TE)—□115 kPa.
   Clinical and radiological features which in the opinion of the investigator correlate with a diagnosis of cirrhosis.
A MELD score (Pre-2016) of [10 and 017 at screening visit.

### Exclusion Criteria

Refusal or inability to give written informed consent to participate in the study.
Other causes of chronic liver disease/cirrhosis not included in the listed aetiologies
Portal hypertensive haemorrhage; active episode of bleeding requiring hospitalisation in the last 3 months where varices have not been eradicated by endoscopic band ligation or transjugular intrahepatic portosystemic shunts (TIPSS).
Ascites unless, in the opinion of the investigator, is minimal and well controlled with no increase to diuretic therapy in the last 3 months.
Hepatic encephalopathy; current or requiring hospitalisation for treatment in the last 3 months.
HCC-uncertain cases to be discussed at the local hepatobiliary multidisciplinary team meeting. Dysplastic or indeterminate nodules to be excluded; regenerative or other nodules to be included at discretion of investigator.
Previous diagnosis of HCC.
Previous organ transplant recipient.
Listed for liver transplantation.
Any situation that in the investigators opinion may interfere with optimal study participation such as alcohol or drug abuse, domicile too distant from study site, potential non-compliance or inability to cooperate.
Presence of clinically relevant acute illness which may preclude on basis of safety.
Presence or history of cancer with exception of adequately treated localised skin carcinoma, in situ cervical cancer or solid malignancy excised in total, with no recurrence (5-year interval).
Pregnancy or breast feeding.

### Interventions

The autologous macrophage therapy was produced in accordance with the method as published in WO2019175595 and Moroni et al. (Nature medicine, 25 (10), 1560-1565).

Isolation of Cells from Patients

PBMCs were collected by leukapheresis from donors who gave informed consent to participate in the study. Leukapheresis of peripheral blood for mononuclear cells (MNCs) was carried out using an Optia apheresis system by sterile collection. A standard collection program for MNC was used, processing 2.5 blood volumes. Isolation of CD 14 cells was carried out using a GMP-compliant functionally closed system (CliniMACS Prodigy system, Miltenyi Biotec). Briefly, the leukapheresis product was sampled for cell count and an aliquot taken for pre-separation flow cytometry. The percentage of monocytes (CD 14+) and absolute cell number were determined, and, if required, the volume was adjusted to meet the required criteria for selection (<20^{c} 10⁹total white blood cells; <400^{c} 10⁶white blood cells/mL; <3.5^{c} 10⁹ CD 14 cells, volume 50-300 mL). CD 14 cell isolation and separation was carried out using the CliniMACS Prodigy with CliniMACS CD 14 microbeads (medical device class III), TS510 tubing set and LP-14 program. At the end of the process, the selected CD14+ positive monocytes were washed in PBS/EDTA buffer (CliniMACS buffer, Miltenyi) containing pharmaceutical grade 0.5% human albumin (Alburex), then re-suspended in TexMACS (or comparator) medium for culture.

### Cell Count

Cell counts of total MNCs and isolated monocyte fractions were performed using a SysmexXP-300 automated analyzer (Sysmex). Assessment of macrophage numbers was carried out by flow cytometry with TruCount tubes (Becton Dickinson) to determine absolute cell number, as the Sysmex consistently underestimated the number of macrophages. The purity of the separation was assessed using flow cytometry (FACSCanto II, BD Biosciences) with a panel of antibodies against human leukocytes (CD45-VioBlue, CD15-FITC, CD14-PE, CD16-APC), and product quality was assessed by determining the amount of neutrophil contamination (CD45int, CD15pos).

### Cell Harvesting

For normal donor-derived macrophages, cells were removed from the wells at day 7 using Cell Dissociation Buffer (Gibco, Thermo Fisher) and a pastette. Cells were resuspended in PEA buffer and counted, then approximately 10⁶ cells per test were stained for flow cytometry. Leukapheresis-derived macrophages were removed from the culture bags at day 7 using PBS/EDTA buffer (CliniMACS buffer, Miltenyi) containing pharmaceutical grade 0.5% human albumin from serum (HAS; Alburex). Harvested cells were resuspended in excipient composed of two licensed products: 0.9% saline for infusion (Baxter) with 0.5% human albumin (Alburex).

Culturing Monocytes from Patient Samples

Monocytes cultured from leukapheresis from Prodigy isolation were cultured at 2^{c} 10⁶ monocytes per cm² and per mL in culture bags (MACS GMP differentiation bags, Miltenyi) with GMP-grade TexMACS (Miltenyi) and 100 ng/ml M-CSF. Monocytes were cultured with 100 ng/ml GMP-compliant recombinant human M-CSF (R&D Systems). Cells were cultured in a humidified atmosphere at 37° C., with 5% C02 for 7 days. A 50% volume media replenishment was carried out twice during culture (days 2 and 4) with 50% of the culture medium removed, then fed with fresh medium supplemented with 200 ng/ml M-CSF (to restore a final concentration of 100 ng/mL).

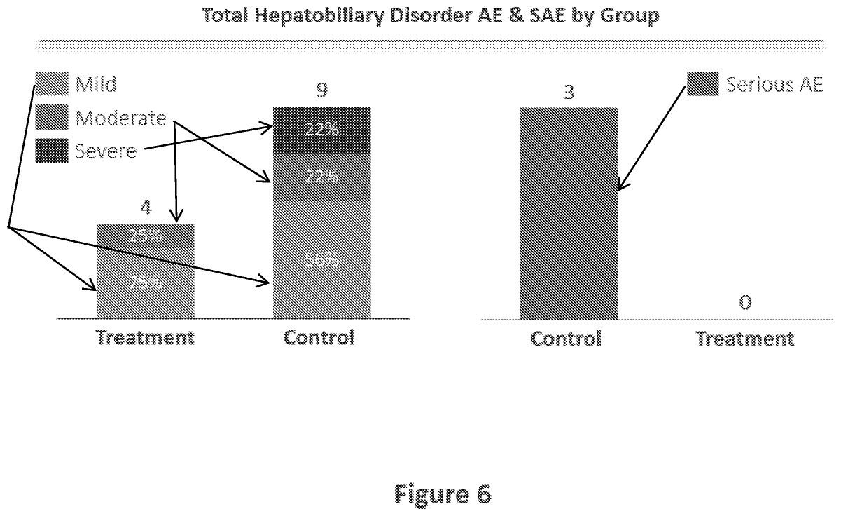

CD 14+ cells were selected using the CliniMACS Prodigy device, and the cells were cultured as described above. The levels of expression of each marker are shown on day 0 enriched monocytes and corresponding day 7 macrophages in FIG. 6. Differentiated cells retain CD45+CD 14+ expression and 25F9, CD206, CD 169 and CD 163 was significantly elevated in macrophages. CCR2 becomes significantly down-regulated in macrophages when compared with monocytes. The migratory capacity of the macrophages post-harvest was also assessed using transwell chemotaxis assay and confirmed that they retained the ability to migrate to suitable targets in vitro despite the down-regulation of CCR2 (data not shown).

### Autologous Unpolarized Macrophage Product

CD 14+ monocytes are isolated from the leukapheresis product utilising the CliniMACS Prodigy® closed system. CD 14+ monocytes are cultured for 7 days in low adhesion culture bags (Miltenyi) in the presence of TexMACS™ serum-free media (Miltenyi) and 100 ng/ml M-CSF (R&D Systems) as described above. A 33% medium change is given at days 3 and 5. In addition, the final product is assessed for markers of macrophage phenotype (25F9 and CD 14) and functional markers (CD206). Assessment of viability is performed by DRAQ7 staining as described above. The final product is harvested and prepared for infusion as a population of <1×10⁷; <1×10⁸ or <1×10⁹ cells in 125 mL 0.9% Saline (Baxter) and 0.5% Human Albumin Solution. The product is presented in a transfer bag (Terumo 150 mL Transfer Bag) and over wrapped prior to transfer to the investigator site for use within 48 hours.

### Treatment

Participants who were randomised to the treatment arm received an infusion of the maximum achieved dose up to 1×10⁹ (day 0). The apheresis product was collected under the terms of the Human Tissue (Quality and Safety for Human Application) Regulations 2007 No. 1523 enacting the requirements of the EU Tissues and cells Directive (2004/2023) and associated Commission Directives at the Apheresis Unit (Royal Infirmary of Edinburgh, Edinburgh, UK). CD14+ monocytes were isolated, and the macrophage cell product was manufactured in compliance with GMP regulations under the terms of the SNBTS MIA (IMP) licence at the SNBTS Cell Therapy Facility (Scottish Centre for Regenerative Medicine, Edinburgh, UK).

Each patient was monitored closely during the infusion to identify potential hypersensitivity reactions and 4 hours postinfusion bloods to monitor for any evidence of macrophage activation syndrome. A total of 28 participants were randomised to standard medical care and 28 to receive the cell infusion, allowing for original estimate of 5 dropouts from each arm. Additional safety data was collected for the first infusion only for the first three patients randomised to the treatment arm. If it was possible to achieve 1×10⁹ macrophages, then the participants were infused with the quantity obtained, with minimum concentration being 1.25×10⁸ cells. This minimum cell concentration was derived from previous validation work and is stipulated as part of the product release criteria as designated by the MHRA.

### Measured Outcomes

### Primary Outcome Measure

### Model of End-Stage Liver Disease

The MELD was originally devised to predict survival in patients with complications of portal hypertension undergoing elective placement of TIPSS. The algorithm is based on: creatinine, bilirubin and prothrombin ratio and has been demonstrated to be superior to the Child-Turcotte-Pugh score in predicting 3-month mortality among patients with end-stage liver disease. However, the MELD score has also been applied to predict survival in patients with cirrhosis with infections, variceal haemorrhage, and those with fulminant hepatic failure and alcoholic hepatitis.

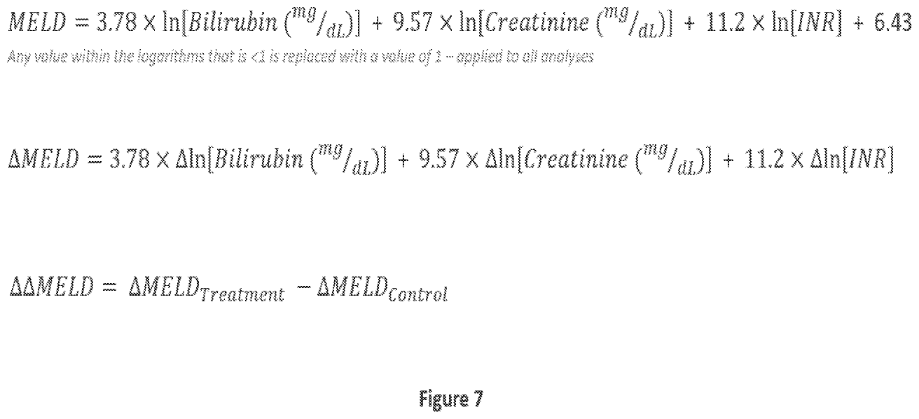

MELD score is calculated using a formula measuring various indications of liver disease (as set forth in FIG. 7). It is regularly used to estimate the chances of a patient with end-stage chronic liver injury surviving their disease (i.e. remaining alive) during the next three months, and thus is often a measurement for prioritizing a patient for liver transplant. A MELD score changes as a patient's disease changes and therefore a change in MELD is a more significant determinant of death and disease than initial MELD alone. The measured change, also termed ΔMELD, is calculating by deducting a patient's prior MELD score from the patient's current MELD score. Therefore, a negative value for ΔMELD may indicate an improvement in clinical status of the patient. Of note, in addition to the MELD, the Sodium MELD score (Na-MELD or MELDNa) can also be used to measure the severity of chronic liver disease.

### Secondary Outcome Measures

### Transplant-Free Interval

The number of participants in each of the two treatment arms who were transplant free at 12 months was recorded.

### Recruitment

### Identification of Potential Patients

Potential participants were identified by their usual direct healthcare team. The treating physician either introduced the individual to the trial team or ask permission for the trial team to contact them. The participant information sheet was provided and there was an opportunity to ask questions. If they agreed, a further visit was scheduled to discuss trial enrolment. This took place no less than 24 hours later.

### Randomisation

Following confirmation of the participant meeting the eligibility criteria, a delegated member of the research team entered minimal information (participant id, and aetiology) into an online randomisation system, produced for the study by Edinburgh Clinical Trials Unit to deter-mine the treatment allocation. At randomisation, patients were allocated a unique patient trial number and scheduled for treatment and follow-up visits as detailed in the trial schedule.

### Allocation

Participants were assigned to receive either standard medical care or to receive a fresh dose of autologous MDMs at the maximum achievable dose, in a 1:1 ratio based on a minimisation algorithm using the key variable aetiology of disease (ALD, NAFLD, other.) To ensure the allocation was random, participants were assigned to the group which minimises the imbalance with probability 0.8. If a participant fell into two or more strata, then the dominant aetiology (as determined by treating physician) was used.

### Blinding

Due to the nature of the intervention neither participants nor staff could be blinded to allocation of treatment.

For some of the additional secondary outcomes we will maintain blinding of external assessors including those processing samples for ELF and protein fingerprint markers. Similarly, there is blinding of MRI physicists and external validation companies responsible for experimental MRI interpretation.

### Data Collection

The case report form (CRF) was completed at set time points as per trial schedule. The CRF was completed by the investigator or an authorised member of the research team (as delegated on the Site Signature and Delegation Log). The exception was the serious AE Form which was signed by the investigator.

### Data Management

The following personal data was collected as part of the research: name, date of birth and CHI numbers (Community Health Index; a unique is a 10-character numeric identifier, allocated to each patient on first registration with the NHS system in Scotland). Personal data was stored in locked cabinets by the research team at the clinical research facilities at each site. Personal data was stored for 30 years in keeping with the blood safety and quality regulations. The University of Edinburgh and NHS Lothian are joint data controllers along with any other entities involved in delivering the study that may be a data controller in accordance with applicable laws.

Study data was collected and managed using Research Electronic Data Capture (REDCap) electronic data capture tools hosted at The University of Edinburgh. REDCap39 is a secure, web-based application designed to support data capture for research studies, providing: an intuitive interface for validated data entry; audit trails for tracking data manipulation and export procedures; automated export procedures for seamless data down-loads to common statistical packages; and procedures for importing data from external sources.

Published results do not contain any personal data that could allow identification of individual participants.

### Example 2. The Macrophage Composition Limits Progression of Chronic Liver Disease in Patients Suffering from Compensated Cirrhosis, as Measured by Stabilization in MELD Score

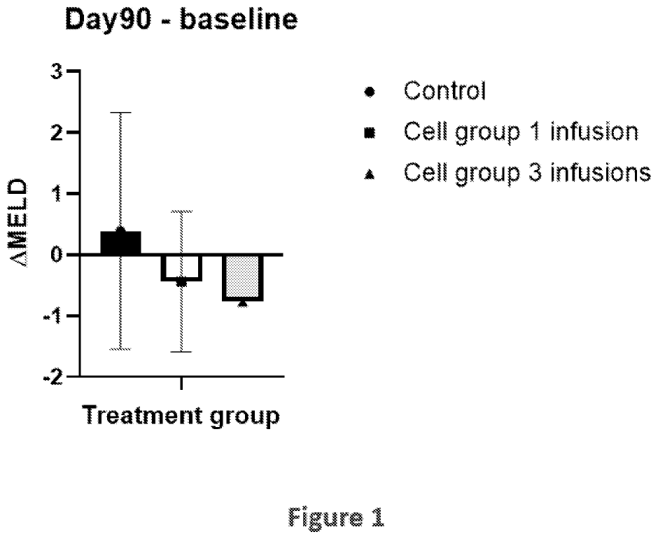

As noted above, from the results of Phase I of the MATCH study (as published in Moroni et al., Nature medicine, 25 (10), 1560-1565 and WO2019175595), the expectation from the MATCH2 study was to see an improvement in MELD score over time as a measurement of improvement in the chronic liver disease (i.e. a reduction in MELD score as expressed by a negative ΔMELD). Indeed, when looking at the average change in MELD score in the MATCH2 study at 90 days (see FIG. 1), the results show that there is on average an improvement in MELD score (i.e. a negative ΔMELD) in the treatment groups as compared to the control group, with a greater improvement in the group receiving 3 infusions. This indicates that the macrophage therapy achieved an improvement in disease symptoms of chronic liver disease (likely associated with liver regeneration and/or resolution of inflammation and fibrosis).

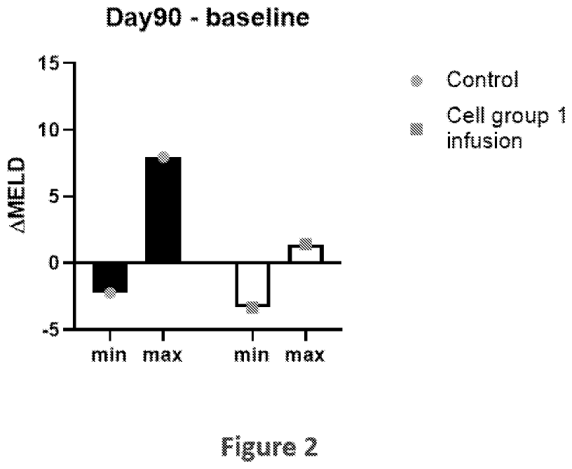

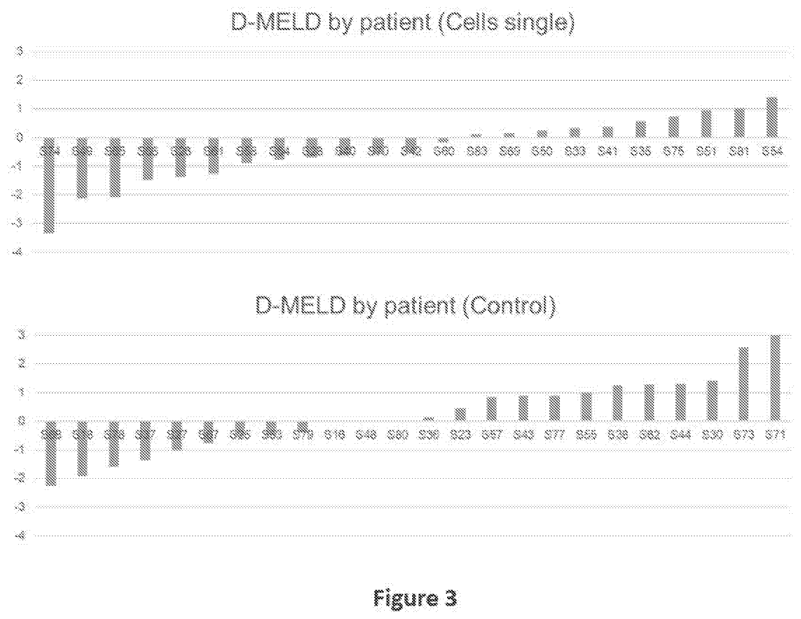

In addition to the improvement in MELD score, an additional and larger unexpected effect was observed, namely a stabilization of MELD scores as a surrogate measurement for limitation of disease progression. As can be seen in FIG. 2 (comparing control patients to patients receiving 1 infusion of the therapeutic macrophages), the ΔMELD score in the patient group was substantially less variable than in the control group (i.e. whereas the ΔMELD in the control group was between −2.25 and 7.59, spanning 5.34 MELD units, the ΔMELD in the patient group was between −3.34 and 1.40, spanning only 1.94 units). In particular, the maximal increase in the ΔMELD score was substantially limited in the patient group compared to the control group (1.40 vs 7.59), with only 1 patient in the treatment group having a MELD score over 1 as opposed to 7 in the control group. This can be seen in FIG. 3. Whereas WO2019175595 suggested that the macrophage treatment may prevent progression of symptoms of liver injury, it was not disclosed or suggested that the macrophage treatment can stabilize liver disease as reflected in MELD score, all the more so limit the progression of MELD scores which may reflect limitation of progression of end stage liver disease. In particular, no limitation in the increase in MELD score at 90 days post infusion was previously suggested such that it only rises by about 1 unit, if at all.

Strikingly, this example demonstrates that the macrophage therapies of the invention are effective for stabilising liver disease and markedly reducing the number of patients experiencing disease progression, in particular more pronounced disease progression. Therefore, the therapies of the invention are effective for reducing the risk of disease progression for each individual patient. Disease progression may be measured by MELD score or delta MELD, indicating a change in MELD score. Liver disease is often a chronic disease with symptoms that can reduce or increase over time with or without treatment.

Although some patients may exhibit a reduction in MELD score (negative delta MELD), even without any treatment, the examples show that the macrophage treatments of the invention are effective for reducing the number of patients experiencing disease progression, and therefore, effective for reducing the risk of disease progression for each individual patient. Therefore, the invention provides a new treatment option, for clinical scenarios wherein any risk of disease progression is unacceptable.

Example 3. The Effect of the Macrophage Composition on MELD Score Lasts for at Least 360 Days

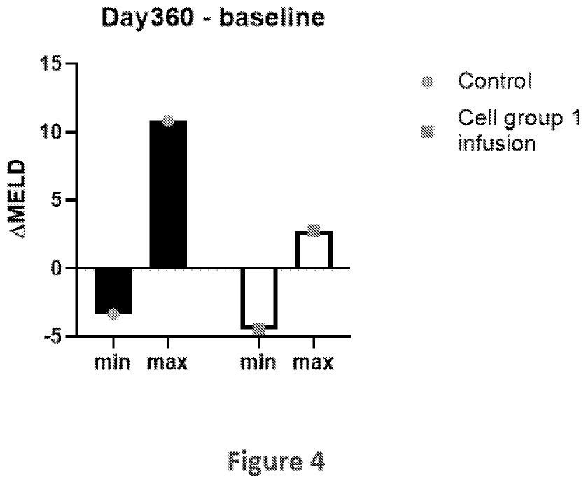

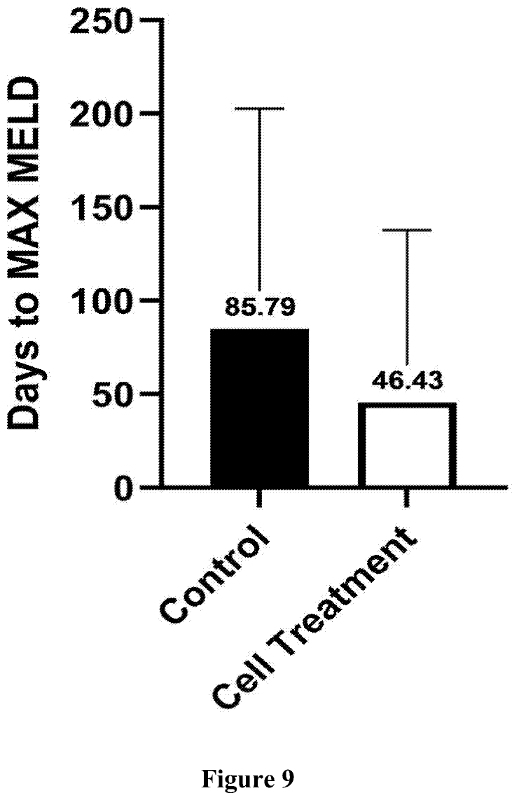

As noted above, the data from Phase 1 of the MATCH study (as shown in FIG. 9 of WO2019175595) suggested that the biggest improvement in disease symptoms (as reflected by ΔMELD score) was 90 days following administration, with an increase in the ΔMELD score after 90 days. However, as can be seen in FIG. 4 (comparing control patients to patients receiving 1 infusion of the therapeutic macrophages), the MATCH2 data surprisingly show that both therapeutic effects are maintained for at least 360 days, namely the average improvement (decrease) in ΔMELD in the treatment group vs the control group, and the limitation in disease progression in the treatment group as reflected by the limitation in increase of ΔMELD.

Importantly, this example demonstrates that the macrophage therapies of the invention are effective for providing therapeutic effects of extended duration. In particular, therapeutic effects including MELD score reductions, limitation of MELD score increases, reduction in decompensation, reduction in deaths, and reduction in adverse hepatobiliary events were observed after 90 days, 360 days and even after extended follow up at 430 days. These results are particularly surprising, considering that administered macrophages would be expected to be cleared from the body within 1-2 weeks of treatment. Furthermore, the therapeutic effect described in WO2019/175595 appears to peak at only 90 days, with delta MELD scores increasing from 90 days (see FIG. 9). The duration of therapeutic effect that is revealed in the examples provides new treatment option for patients with liver disease. Alternative therapies may be intended to provide some relief from symptoms or disease progression over a short period, until a suitable transplant can be arranged. The present invention allows patients with liver disease to be treated for longer periods.

### Example 4. The Effect of the Macrophage Composition on Clinical Events Lasts for at Least 360 Days

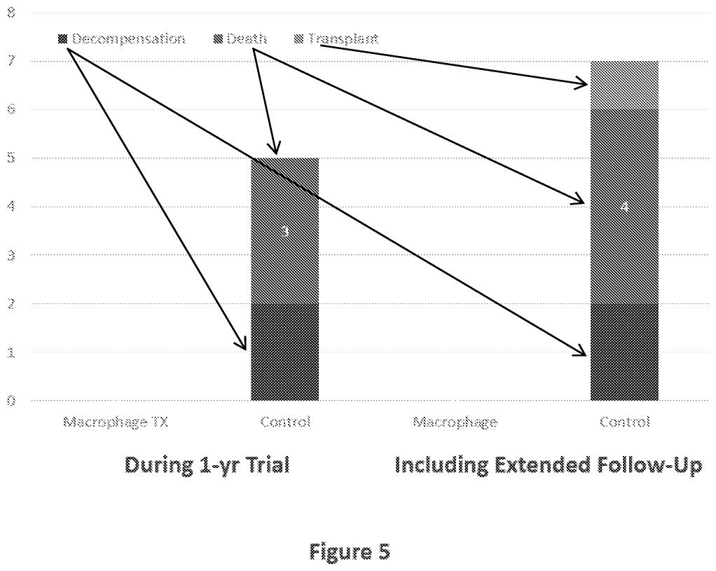

Furthermore, within 360 days following administration of the cell composition to the patients, there were no deaths or major liver-disease associated events (liver decompensation, death or liver transplant) in the treatment group. Conversely, within the study period there were in the control group 5 major clinical events-3 deaths (1 of which is of a patient who withdrew before the end of the study) and 2 events of liver decompensation which are also considered Serious Adverse Events (SAEs) (see FIG. 5, left-hand side). In the extended follow-up period of 460 days after infusion, while there were still no deaths or major liver-associated clinical events in the treatment group, there were 2 additional events in the control group—an additional death and a transplant (FIG. 5, right-hand side), bringing the number of liver-related major clinical events to 7. When looking at liver-related Adverse Events (AEs), there were 9 AEs in the control group (3 of which are SAEs-2 decompensation events and 1 ESRD) and only 4 AEs in the treatment group (with no liver-related SAEs). The left-hand side of FIG. 6 depicts the liver-related AEs by degree of severity.

Strikingly, this example also demonstrates that the macrophage therapies of the invention are effective for preventing decompensation. In particular, no patients receiving the macrophage therapy suffered decompensation, either over a year or after extended follow-up. In contrast, 2 decompensation events were observed in the control group.

Furthermore, this example demonstrates that the macrophage therapies of the invention are effective for reducing the risk of end stage renal disease. In particular, no patients receiving the macrophage therapy developed end-stage renal disease during the year-long trial or after extended follow-up. In contrast, end stage renal disease was observed in the control group.

Significantly, this example also demonstrates that the macrophage therapies of the invention are effective for reducing adverse hepatobiliary events. In particular, only 4 patients receiving the macrophage therapy experienced adverse events, and there were no serious events. In contrast, 9 adverse events, 3 of which were serious, were observed in the control group.

These data also indicate that the treatment limits disease progression for at least 360 days, which was surprising both in view of the results of Phase 1 of the MATCH data and in view of pharmacokinetics experiments in mice which suggest that the macrophage composition is cleared from the body within 1-2 weeks of treatment (data not shown), making this long-term efficacy particularly striking. The striking potency of the therapies tested in the examples provides a new treatment for end-stage liver disease, which otherwise has no treatment available. In addition, the duration of effect that is revealed in the examples provides a new treatment option for liver disease and end-stage liver disease in particular, because they can be treated without transplant for significant periods of time. Accordingly, the macrophage therapy of the present invention has the ability to stabilise patients with liver disease, and it therefore uniquely placed to treat those who may have an extended wait for a transplant.

Overall, the examples demonstrate that the macrophage therapies of the invention are effective for stabilising liver disease and markedly reducing the number of patients experiencing disease progression, in particular more pronounced disease progression. Therefore, the therapies of the invention are effective for reducing the risk of disease progression for each individual patient. Disease progression may be measured by MELD score or delta MELD, indicating a change in MELD score. Liver disease is often a chronic disease with symptoms that can reduce or increase over time with or without treatment. Although some patients may exhibit a reduction in MELD score (negative delta MELD), even without any treatment, the examples show that the macrophage treatments of the invention are effective for reducing the number of patients experiencing disease progression, and therefore, effective for reducing the risk of disease progression for each individual patient. Therefore, the invention provides a new treatment option, for clinical scenarios wherein any risk of disease progression is unacceptable.

While the results set out in the above examples were obtained using an unpolarised macrophage product, M2-like cells as described herein would be expected to be similarly, if not more, effective. As explained herein, chronic liver injury models illustrate the role of proinflammatory cells in promoting fibrogenesis, and that repair is associated with a switch to an anti-inflammatory, M2-like phenotype. M2-like macrophages would be expected to be particularly efficacious in promoting liver repair due to the enhanced expression of matrix metalloproteinases (MMPs), growth factors and phagocytosis-related genes to resolve pathological inflammation. Accordingly, polarised, M2-like cells would be expected to show even greater efficacy in treating liver disease in a patient.

### Example 5—the MELD Score of Patients Treated with the Macrophage Composition Stabilises More Quickly than Untreated Patients

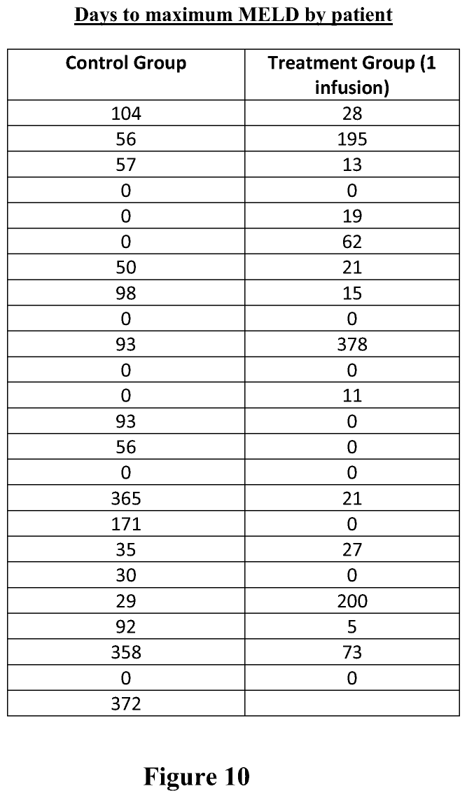

In order to further investigate the stabilisation effect of the therapeutic macrophages, the data generated by the trial whose protocol is set out in Example 1 was further analysed to determine the length of time for each patient to reach the maximum MELD score. The mean number of days taken for treated (1 infusion) and control patients to reach the maximum MELD score are shown in FIG. 9, and the individual times for patients to reach the maximum recorded MELD score are set out in FIG. 10. As demonstrated in the data in FIG. 9, patients treated with 1 infusion of the macrophage therapy reached the maximum MELD score more quickly than those in the control group. Therefore, not only do the therapeutic macrophages reduces the risk and extent of disease progression, but they also reduce the length of time over which patients deteriorate. Accordingly, it can be seen from the data in FIG. 9 that patients treated with the macrophage therapy stabilise more quickly than those untreated. The therapeutic macrophages may therefore be particularly useful to treat patients suffering from liver cirrhosis that have undergone a first hepatic decompensation event requiring hospitalisation, and can be used to stabilise the patients before any further hepatic decompensation events occur.

### Example 6-3 Year Follow Up Data Shows that the Macrophage Composition Increases Long Term Survival, Reduces all-Cause Mortality and Reduces Need for Liver Transplant

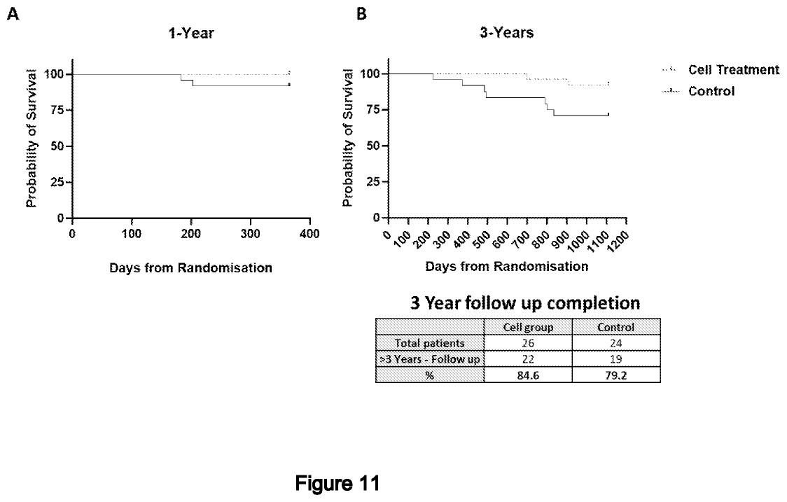

In order to investigate the long term effect of the therapeutic macrophages, long follow up data of the patients described herein above was analysed. Kaplan-Meier survival analysis, a non-parametric statistical method, was employed to assess the differences in survival between the treatment and control groups from time-to-event data. The method generates a survival plot for each group under study, with the survival function assumed to be constant between successive distinct sampled observations. One survival plot was generated for control and cell treatment groups, each plot. For this, two analyses were performed, the first one compromising on the complete 1-year follow-up dataset (FIG. 11A) and a non-completed 3-year follow-up dataset (FIG. 11B).

For both analysis, cell treatment included both one and three dose treatments, this was done to increase the n number and to investigate the effect of a macrophage cell therapy versus control regarding of the number of doses administered. For the 1-year analysis, all patients were considered to reach 365 days survival unless death was recorded. For the 3-year follow up, patient percentage completion of the follow-up was 84.6% and 79.25% for cell treatment and control respectively as per January 2024. All patients that completed the follow up were considered to reach 1110 days survival unless death was recorded. Patients that have not reached 3-years follow up, a 1110 days survival was assumed for this analysis. The Log-rank (Mantel-Cox) test was performed, this analysis uses proportional hazards for survival, meaning that the ratio of hazard functions (deaths per time) is the same at all time points. All data analysis was completed using GraphPad Prism, Version 10.1.12.

TABLE 1
Log-rank (Mantel-Cox) test summary for 1
and 3 year Kaplan Meier survival graphs.

| | |
|---|---|
| Log-rank | Comparison of Survival Curves |

| (Mantel-Cox) test | 1 Year | 3 Years |
|---|---|---|
| Chi square | 2.213 | 4.091 |
| P value | 0.1368 | 0.0431 |
| P value summary | ns | * |
| Are the survival curves | No | Yes |
| significantly different? | | |

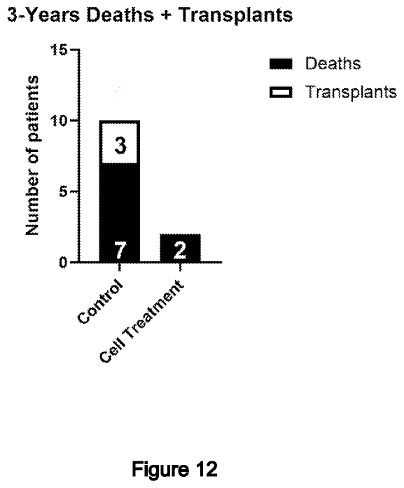

As can be seen in FIG. 11A, Kaplan-Meier survival analysis at one year did not show any significant difference on the 1-year between treatment and control. However, at 3-years (Table 1 and FIG. 11B), comparison on survival curves by Log-rank (Mantel-Cox) test did show a significant difference (p=0.0431) between treatment or control. In addition, as can be seen in FIG. 12, when looking at all-cause mortality as well as transplants between control and cell treatment, there is a reduction of all-cause mortality as well as no transplants on the cell therapy group within the 3 year period. Kaplan-Meier survival analysis at 3-years highlights the effectiveness of macrophage cell therapy for ESLD in terms of increased survival and reduction of transplants in the treatment group.

### Example 7-Treatment with the Macrophage Composition does not Induce an Increase in Proinflammatory or Pathogenic Cytokines for at Least 360 Days

To confirm that the macrophage composition is well tolerated in patients with liver cirrhosis, a pro-inflammatory marker analysis was performed using Meso Scale Discovery (MSD). To aid in defining patient tolerance to macrophage cell therapy, serum cytokines were analysed using a V-PLEX Proinflammatory Panel and a V-PLEX Cytokine Panel according to the manufacturers' instructions (Meso Scale Discovery, Meso Scale Diagnostics, LLC.). A set of four safety-related cytokines were selected which are associated with ‘cytokine release syndrome (CRS)’ in MAS. These were IL-8 (pivotal in the pathogenesis of MAS), IL-1B, IL-6 and TNF-α. Following quantification, differences in inflammatory marker concentration between treatment groups were inferred via two-way ANOVA with post-hoc comparisons using Dunnett's method—where the data frame contained missing values, a mixed effects model was utilised. All data analysis was completed using GraphPad Prism, Version 10.1.12.

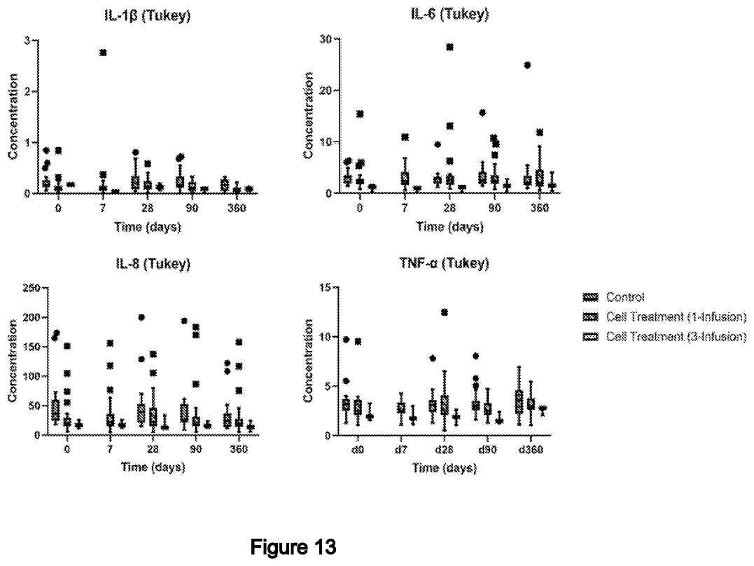

As can be seen in FIG. 13, based on the data herein, no statistically significant differences in the concentration of circulating proinflammatory or pathogenic cytokines were observed following macrophage cell therapy infusion after 360 days of monitoring, suggesting a lack of MAS and CRS following systemic administration of macrophage cell therapy, and thereby providing confirmatory evidence that this class of therapy is well tolerated in patients with liver cirrhosis.

### Example 8-Full Time dMELD Analysis

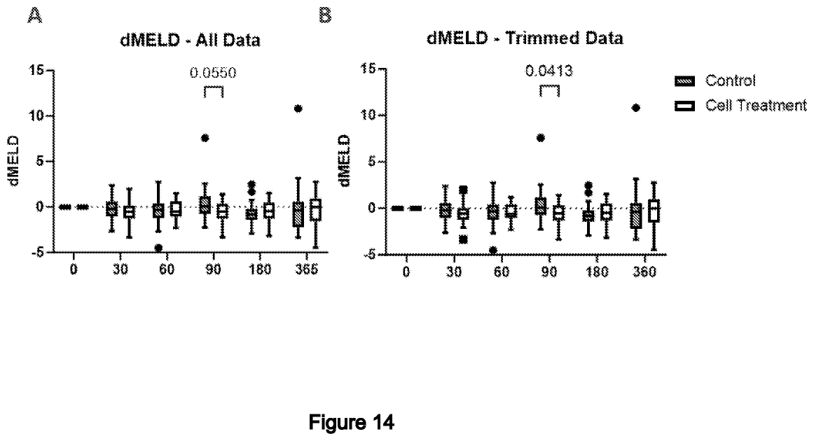

An additional analysis on dMELD score was performed to investigate dynamics of dMELD over the 1-year follow up study. For this, comparison of control versus cell treatment, single dose, was performed (FIG. 14A). A sub-group analysis was also performed (FIG. 14B) when a patient on the treatment group was excluded due to relapse of their alcohol misuse disorder during the trial. Analysis of all timepoints allows the generation of a model that analyse variations between groups over time, providing a holistic view of the dataset.

Multiple comparison of both timepoints and treatment was done for the additional dMELD analysis. For the analysis, a mixed-effect model with a Tukey multiple comparison test was performed. A sub-group analysis was performed (FIG. 14B) for the reasons outlined above. All data analysis was completed using GraphPad Prism, Version 10.1.12.

The additional analysis on dMELD highlighted the effect of dMELD reduction at 90 days. More importantly, when looking at the subgroup where a patient was excluded due to relapse of their alcohol misuse, significant reduction (p=0.0413) of dMELD at 90 days was observed. This analysis further emphasises the effects of macrophage cell therapy on MELD score reduction, indicating an improvement of liver function.

1. A composition comprising macrophages, for use in the treatment of liver disease in a patient, wherein the treatment provides a greater reduction in symptoms than a treatment that does not comprise administration of macrophages.

2. The composition for use of claim 1, wherein the composition is for use in the treatment of inflammatory liver injury, such as inflammatory liver injury with a fibrotic element, such as chronic liver injury, such as cirrhosis.

3. The composition for use of claim 1, wherein the composition is for use in preventing decompensation in a patient with compensated liver cirrhosis.

4. The composition for use of claim 1, wherein the composition is for use of reducing the risk of one or more major clinical complications associated with liver cirrhosis, optionally major clinical complications of liver cirrhosis selected from the group consisting of: ascites, hepatic encephalopathy, gastrointestinal variceal haemorrhage, spontaneous bacterial peritonitis, jaundice, hepatorenal syndrome, gastrointestinal haemorrhage, death or a combination thereof.

5. The composition for use of claim 4, wherein the composition is for use in reducing the risk of death for a patient with liver disease.

6. The composition for use of claim 5, wherein the composition is for use in reducing the risk of death over a period of 3, 6, 9, 12, 14, 18, 24 or 36 months following final administration of the composition.

7. The composition for use of claim 1, wherein the composition is for use in reducing the risk of end stage renal disease in a patient with liver disease.

8. The composition for use of claim 1, wherein the composition is for use in reducing adverse hepatobiliary events in a patient with liver disease, such as serious adverse hepatobiliary events.

9. The composition for use of claim 1, wherein the treatment reduces the risk of disease progression.

10. The composition for use of claim 1, wherein the treatment is for slowing disease progression, optionally wherein slowing disease progression is measured by delay in increase in MELD score.

11. The composition for use of claim 9 or 10, wherein the treatment reduces the risk of disease progression over a period of 3, 6, 9, 12, 14, 18 or 24 months following final administration of the composition.

12. The composition for use of claims 9-11, wherein disease progression is measured by a change in MELD score.

13. The composition for use according to claim 12, wherein treatment limits the total change in MELD score for the patient at 90 days to a total change of less than 2 units.

14. The composition for use of claim 12 or 13, wherein the treatment reduces the risk of the MELD score for the patient increasing by 1 or more, 1.5 or more, or 2 or more, optionally over a period of 3, 6, 9, 12, 14, 18 or 24 months following final administration of the composition.

15. The composition for use of claims 9-14, wherein the treatment prevents disease progression over a period of 3, 6, 9, 12, 14, 18 or 24 months following final administration of the composition.

16. The composition for use of any preceding claim, wherein the treatment reduces all-cause mortality for a period of at least 1 year, optionally at least 2 years, preferably at least 3 years.

17. The composition for use of any preceding claim, wherein the treatment reduces need for liver transplant for a period of at least 1 year, optionally at least 2 years, preferably at least 3 years.

18. The composition for use of any preceding claim, wherein the liver disease is liver cirrhosis.

19. The composition for use of claim 18, wherein the liver cirrhosis resulted from at least one disease or condition selected from the group consisting of: non-alcoholic fatty liver disease (NAFL) (e.g., non-alcoholic fatty liver disease (NAFLD) or non-alcoholic steatohepatitis (NASH)), alcoholic liver disease (e.g., alcoholic fatty liver disease (AFLD) or alcoholic steatohepatitis (ASH)), mechanical trauma to the liver, biliary obstruction, autoimmune hepatitis, iron overload, Hepatitis B infection (HBV) and Hepatitis C infection (HCV).

20. The composition for use of claim 18, wherein the liver cirrhosis resulted from steatotic liver disease (SLD), optionally wherein the steatotic liver disease is metabolic dysfunction-associated steatotic liver disease, metabolic-associated steatohepatitis, Met-ALD or Cryptogenic SLD.

21. The composition for use of claims 18-20, wherein the liver cirrhosis is selected from compensated cirrhosis and decompensated cirrhosis.

22. The composition for use of claims 18-21, wherein the patient has recovered from their first hepatic decompensation event (recompensated), optionally wherein the first hepatic decompensation event required the patient's hospitalization, optionally wherein the patient has not undergone an additional hepatic decompensation event after having recovered from the first decompensation event.

23. The composition for use of claims 1-22, wherein the composition is for use in preventing further hepatic decompensation events in a patient who has undergone and recovered from their first hepatic decompensation event, optionally wherein the first hepatic decompensation event required the patient's hospitalization, optionally wherein the patient has not undergone an additional hepatic decompensation event after having recovered from the first decompensation event.

24. The composition for use of claims 18-23, wherein the subject exhibits and/or has recovered from one or more clinical signs of hepatic decompensation selected from the list consisting of jaundice, ascites, hepatic encephalopathy, hepatorenal syndrome, variceal haemorrhage and gastrointestinal haemorrhage.

25. The composition for use of any preceding claim, wherein the liver disease is chronic liver disease.

26. The composition for use of any preceding claim, wherein the liver disease is end-stage liver disease, optionally wherein the end-stage liver disease is liver cirrhosis, optionally wherein the liver cirrhosis is compensated or decompensated liver cirrhosis, optionally wherein the composition is for use in reducing the risk of death for a patient with end-stage liver disease over a period of 3, 6, 9, 12, 14, 18 or 24 months following final administration of the composition.

27. The composition for use of any preceding claim, wherein the liver disease is alcohol-related liver disease or NAFLD.

28. The composition for use of any preceding claim, wherein the patient is scheduled to receive a liver transplant not sooner than 3, 6, 9, 12, 14, 18 or 24 months in the future following final administration of the composition.

29. The composition for use of any preceding claim, wherein the treatment comprises administering the composition and at least 3 months later, such as 6, 9, 12, 14, 18 or 24 months later, providing a liver transplant.

30. The composition for use of any preceding claim, wherein the treatment comprises administration of at least one dose of the composition, such as at least two or at least three doses of the composition, or such as not more than one or not more than two or not more than three doses of the composition.

31. The composition for use of any preceding claim, wherein the macrophages are unpolarised macrophages, such as unpolarised human autologous macrophages.

32. The composition for use of any preceding claim, wherein the macrophages are polarized macrophages, such as polarized human autologous macrophages, such as macrophages that exhibit a pro-restorative M2-like phenotype.

33. The composition for use of any preceding claim, wherein the macrophages are monocyte derived macrophages (MDMs) or iPSC-derived macrophages.

34. The composition for use of any preceding claim, wherein the macrophages are monocyte-derived.

35. The composition for use of any preceding claim, wherein the macrophages are mature.

36. The composition for use of any preceding claim, wherein the composition comprises at least 1×10⁷ macrophages, such as approximately 1×10⁸ or 1×10⁹ macrophages per dose.

37. The composition for use of any preceding claim, wherein treatment with the composition stabilises the patient with liver disease, optionally wherein stabilisation is determined by measuring the MELD score of the patient.

38. The composition for use of claim 37, wherein the patient's MELD score has been determined at regular intervals, and the patient is deemed stabilised when at least 3 consecutive measurements give a MELD score which does not change significantly, optionally wherein the MELD score is determined at monthly intervals.

39. The composition for use of claim 37 or 38, wherein the patient is deemed stabilised when the patient's MELD score does not change significantly for a period of at least 12 months, optionally wherein the MELD score is determined at monthly intervals.

40. The composition for use of claims 37-39, wherein the patient is stabilised more rapidly than an individual untreated with the composition.

41. The composition for use of claims 37-40, wherein the patient is stabilised within 50 days of administration of the composition.
